(19) United States
(12) Patent Application Publication
(10) Pub. No.: US 2023/0173070 A1
(43) Pub. Date: Jun. 8, 2023

(54) METHODS AND SYSTEMS FOR ENHANCING DELIVERY OF THERAPEUTIC AGENTS TO BIOFILMS USING LOW BOILING POINT PHASE CHANGE CONTRAST AGENTS

(71) Applicants: The University of North Carolina at Chapel Hill, Chapel Hill, NC (US); The Regents of the University of Colorado, Denver, CO (US)

(72) Inventors: Virginie Papadopoulou, Chapel Hill, NC (US); Paul Alexander Dayton, Carrboro, NC (US); Sarah Elizabeth Conlon, Durham, NC (US); Brian Patrick Conlon, Durham, NC (US); Phillip Gregory Durham, Chapel Hill, NC (US); Mark A. Borden, Boulder, CO (US)

(21) Appl. No.: 17/922,522

(22) PCT Filed: Jun. 1, 2021
(86) PCT No.: PCT/US2021/035212

§ 371 Date: Oct. 31, 2022

Related U.S. Application Data
(60) Provisional application No. 63/032,905, filed on Jun. 1, 2020.

(51) Int. Cl. A61K 41/00; A61M 37/00; A61K 33/00; A61K 47/26; A61K 47/02; A61K 31/702; A61K 38/12; A61K 31/5377; A61K 31/351; A61K 31/538; A61K 31/7036; A61K 31/496; A61K 38/15; A61K 47/12

(52) U.S. Cl. CPC A61K 41/0047; A61M 37/0092; A61K 33/00; A61K 47/26; A61K 47/02; A61K 31/702; A61K 38/12; A61K 31/5377; A61K 31/351; A61K 31/538; A61K 31/7036; A61K 31/496; A61K 38/15; A61K 47/12; A61M 2202/0208; A61M 2037/0007

(57) ABSTRACT

A method for applying ultrasound to activate a cavitation enhancing agent in the presence of a therapeutic compound and a microbial biofilm is provided. The ultrasound energy causes the cavitation enhancing agent to cavitate in the ultrasound field. The cavitation of the resultant bubble causes fluid streaming and shear forces at and near the biofilm, causing enhanced penetration of the therapeutic compound into the biofilm, and resulting in improved efficacy of the therapeutic compound against the biofilm. The method further includes cavitation enhancing agents which can be loaded with oxygen gas or combined with microbubbles which carry oxygen gas, which further potentiate antibiotic efficacy against the biofilm.

### PRIORITY CLAIM

This application claims the priority benefit of U.S. Provisional Patent Application No. 63/032,905, filed Jun. 1, 2020, the disclosure of which is incorporated by reference herein in its entirety.

### GOVERNMENT INTEREST

This invention was made with government support under Grant Numbers CA206939, CA232148, and AI137273 awarded by the National Institutes of Health. The government has certain rights in the invention.

### TECHNICAL FIELD

The subject matter described herein relates to applying therapeutic agents to biofilms. More particularly, the subject matter described herein relates to method and systems for enhancing delivery of therapeutic agents to biofilms using low boiling point phase change contrast agents.

### BACKGROUND

Biofilms are aggregates of bacterial cells from one or more organisms embedded in a self-produced extracellular matrix and attached to a surface, such as host tissue. Microorganisms that make up biofilms can include bacteria, fungi, and protists. Biofilms are resistant to therapeutic agents, such as antibiotics, because biofilms are formed of multiple layers of microorganisms encapsulated in a polysaccharide matrix, and it is difficult for the therapeutic agent to penetrate the polysaccharide matrix and to reach the deeper layers of microorganisms. In addition, the deeper layers of a biofilm are often oxygen or nutrient deplete environments, resulting in a low metabolic state and making therapeutic agents less effective.

Phase change contrast agents (PCCAs) are particles that are activated by ultrasound for imaging and therapeutic purposes. Phase change contrast agents, such as dodecafluoropentane, have high (>25° C. at atmospheric pressure) boiling points and/or have peak negative pressures on the order of megaPascals for vaporization. The use of phase change contrast agents with high boiling points and/or high peak negative pressures to disrupt biofilms in vivo may have undesirable bioeffects, such as cell lysis, on tissue adjacent to the biofilm being treated. In addition, conventional therapies using PCCAs in combination with therapeutic agents to treat biofilms have not resulted in total eradication of the microorganisms within the biofilm.

Accordingly, in light of these and other difficulties, there exists a need for methods and systems for enhancing delivery of therapeutic agents to biofilms using low boiling point phase change contrast agents.

### SUMMARY

A method of enhancing delivery of a therapeutic agent into a microbial biofilm includes administering a cavitation enhancing agent into the microbial biofilm. The method further includes exposing the microbial biofilm to at least one therapeutic agent. The method further includes delivering ultrasound pulses to the microbial biofilm which cause the cavitation enhancing agent to cavitate; and increase penetration of the at least one therapeutic agent into the biofilm, wherein the cavitation enhancing agent comprises a phase change contrast agent comprising a core including a material that has a boiling point less than 25° C. at atmospheric pressure.

In one example, the microbial biofilm is located in or on the body of a living subject, such as a mammalian subject, including, but not limited to a mouse or a human subject.

### BRIEF DESCRIPTION OF THE DRAWINGS

FIGS. 1A-1C illustrate that PCCA and ultrasound disrupts biofilm and increases drug penetration. FIG. 1A illustrates a nanoscale PCCA in a stable liquid phase. When exposed to ultrasound, the lipid shell containing superheated liquid perfluorocarbon is destabilized, causing the liquid to vaporize (acoustic droplet vaporization, ADV) to the gas phase and expand into a microbubble. FIG. 1B is a schematic diagram of an experimental setup for in vitro ultrasound exposure. An arbitrary waveform generator is used to generate a 1 MHz sine wave which is amplified and transmitted to an ultrasound transducer which is positioned over a bacterial biofilm in a well plate. The well plate is positioned in a custom fabricated water bath and coupled to water maintained at 37° C. The bottom of the water bath is lined with ultrasound absorber material to reduce acoustic reflections. A lid with circular holes is used to center the ultrasound transducer within each well at a consistent height. FIG. 1C illustrates the stability and small size of PCCAs makes them ideal to diffuse into biofilms prior to ultrasound application. Ultrasound stimulation can vaporize PCCAs to microbubbles that can physically disrupt biofilms and enhance drug penetration;

FIG. 2A is a graph of colony forming units (CFUs) per milliliter on a logarithmic scale for an untreated MRSA biofilm and MRSA biofilms treated with different antibiotics but without phase change contrast agents;

FIG. 2B is a graph of CFUs per milliliter on a logarithmic scale for MRSA biofilms treated with tobramycin (TOB), with and without phase change contrast agents, with and without ultrasound, and with ultrasound at different pressures;

FIG. 2C is a graph of CFUs per milliliter on a logarithmic scale for MRSA biofilms treated with different combinations including ultrasound, a PCCA, mupirocin (MUP), vancomycin (VAN), and linezolid (LIN);

FIG. 3A is a graph of CFUs per milliliter on a logarithmic scale for MRSA biofilms treated with different antibiotics combined with PCCAs and ultrasound at different pressures;

FIG. 3B is a top view of a sample illustrating experiments involving the use of combinations of US and a PCCA with anti-persister antibiotic therapy against MRSA biofilms;

FIG. 3C is a graph illustrating results of the experiments illustrated in FIG. 3B;

FIGS. 4A-4C illustrate results of applying US-PCCA with anti-persister drugs to MRSA biofilms;

FIG. 5 is a schematic diagram illustrating a dual approach to improving antibiotic treatment of S. aureus biofilms;

FIG. 6 is a flow diagram illustrating an exemplary process for treating biofilm infections in vitro;

FIG. 7 is a diagram illustrating an exemplary setup for treating biofilm infections in vitro;

FIG. 8 is a diagram illustrating an exemplary system for treating microbial biofilm infections in vivo using an ultrasound transducer, a phase change contrast agent, and a therapeutic agent;

FIG. 9 is a graph illustrating CFUs per milliliter on a logarithmic scale for MRSA biofilms treated with the TOB antibiotic in combination with oxygen nanodroplets and ultrasound at different pressures;

FIG. 10 is a graph illustrating CFUs per milliliter for MRSA biofilms treated with rhamnolipid nanodroplet PCCAs;

FIGS. 11A-11C are, respectively, a schematic diagram and graphs illustrating an experiment and results of the experiment where a phase change contrast agent, an antibiotic, and an antibiotic adjuvant were used to treat an MRSA biofilm in vivo;

FIG. 12 is a graph of results for an experiment where a phase change contrast agent, an antibiotic, and an antibiotic adjuvant were used to treat an MRSA biofilm in vitro;

FIG. 13 is a flow chart illustrating an exemplary process for treating a microbial biofilm infection with a phase change contrast agent, a therapeutic agent, and ultrasound energy;

FIG. 14 is a diagram illustrating a topical treatment device for treating a microbial biofilm infection using a combination of a phase change contrast agent, a therapeutic agent, and ultrasound energy;

FIG. 15 is a diagram illustrating an intravascular treatment device for treating a microbial biofilm infection using a combination of a phase change contrast agent, a therapeutic agent, and ultrasound energy; and

FIG. 16 is a diagram illustrating an endoscopic treatment device for treating a microbial biofilm infection using a combination of a phase change contrast agent, a therapeutic agent, and ultrasound energy.

### DETAILED DESCRIPTION

Bacterial biofilms, often associated with chronic infections, respond poorly to antibiotic therapy and frequently require surgical intervention. Biofilms harbor persister cells, metabolically indolent cells, which are tolerant to most conventional antibiotics. In addition, the biofilm matrix can act as a physical barrier, impeding diffusion of antibiotics. Novel therapeutic approaches frequently improve biofilm killing, but usually fail to achieve eradication. Failure to eradicate the biofilm leads to chronic and relapsing infection, associated with major financial healthcare costs and significant morbidity and mortality. We address this problem with a two-pronged strategy using 1) antibiotics that target persister cells and 2) ultrasound-stimulated phase-change contrast agents (US-PCCA), which improve antibiotic penetration.

We previously demonstrated that rhamnolipids, produced by Pseudomonas aeruginosa, could induce aminoglycoside uptake in gram-positive organisms, leading to persister cell death. We have also shown that US-PCCA can transiently disrupt biological barriers to improve penetration of therapeutic macromolecules. We hypothesized that combining antibiotics which target persister cells with US-PCCA to improve drug penetration could improve treatment of methicillin resistant S. aureus (MRSA) biofilms.

Aminoglycosides alone or in combination with US-PCCA displayed limited efficacy against MRSA biofilms. In contrast, the anti-persister combination of rhamnolipids and aminoglycosides combined with US-PCCA dramatically improved using a combined approach of improving drug penetration of therapeutics that target persister cells. This novel treatment strategy has the potential for rapid clinical translation as the PCCA formulation is a variant of FDA-approved ultrasound contrast agents that are already in clinical practice and the low-pressure ultrasound settings used in our study can be achieved with existing ultrasound hardware at pressures below the FDA set limits for diagnostic imaging.

### Introduction

S. aureus is one of the most important human bacterial pathogens and in 2017 was the cause of 20,000 bacteremia deaths in the US alone¹. Infections range from minor skin and soft tissue infections (SSTI), implanted device infections to more serious infections such as osteomyelitis, endocarditis and pneumonia^{2,3}. In addition to the high degree of mortality, chronic and relapsing S. aureus infections are common and associated with significant morbidity. This is due to frequent treatment failure of S. aureus infections. This is best illustrated by SSTIs, with some studies suggesting treatment failure rates as high as 45% and a recurrence rate of 70%⁴. Importantly the failure of antibiotic therapy cannot be adequately explained by antibiotic resistance¹. Failure to clear the infection leads to a need for prolonged antibiotic therapies, increased morbidity and mortality, increased likelihood of antibiotic resistance development as well as an enormous financial healthcare burden.

S. aureus forms biofilms, bacterial cells embedded in a self-produced extracellular matrix, which act as a protective barrier from the host immune response and other environmental assaults. Biofilms expand up to 1200 μm in thickness when attached to indwelling devices such as catheters⁵. Non-surface attached biofilms in chronic wounds and chronic lung infections harbor smaller, non-surface attached cell aggregates ranging from 2-200 μm in diameter^{5,6}. These biofilm aggregates are often surrounded by inflammatory immune cells such as neutrophils and embedded in a secondary host produced matrix such as mucus, pus or wound slough⁷. Consequently, biofilm-embedded cells have limited access to nutrients and oxygen and are coerced into a metabolically indolent state⁸.

It has long been appreciated that biofilms respond poorly to antibiotics^{7,9-12}. Most conventional bactericidal antibiotics kill by corrupting ATP-dependent cellular processes; aminoglycosides target translation, fluoroquinolones target DNA synthesis, rifampicin targets transcription and β-lactams and glycopeptides target cell wall synthesis^{13,14}. Cells that survive lethal doses of antibiotics in the absence of a classical resistance mechanism are called antibiotic tolerant persister cells¹⁵. Biofilms are made up of a high proportion of persister cells¹⁵⁻¹⁸. They are distinct from resistant cells as they cannot grow in the presence of the drug. However, once the drug is removed, persisters grow and repopulate a biofilm and cause a relapse in infection¹³. Anti-persister antibiotics which kill independently of the metabolic state of the cell are more effective against biofilms than conventional antibiotics¹⁹⁻²². Tobramycin, an aminoglycoside that requires active proton motive force (PMF) for uptake into the cell is inactive against non-respiring cells, anaerobically growing cells, small colony variants and metabolically inactive cells within a biofilm²⁰. We previously reported that rhamnolipids, biosurfactants produced by P. aeruginosa, permeabilize the S. aureus membrane to allow PMF-independent diffusion of tobramycin into the cell^{20,22}. This combination of tobramycin and rhamnolipids (TOB/RL) rapidly sterilized in vitro planktonic cultures as well as non-respiring cells, anaerobically growing cells and small colony variants. However, despite this potent anti-persister activity, TOB/RL reduced biofilm viability by ˜3-logs but failed to achieve eradication²⁰. Notwithstanding the promise of this strategy, eradication of biofilms is arduous, even in vitro, indicating that factors other than the metabolic state of the biofilm-embedded cells are impeding therapy.

The biofilm matrix can act as a physical barrier to drug penetration. Penetration of vancomycin, β-lactams, phenicols and aminoglycoside antibiotics are impeded to some extent into S. aureus biofilms²³⁻²⁶. Consequently, novel methods of drug delivery into biofilms is a growing area of interest. Ultrasound is a safe, commonplace, portable and relatively inexpensive modality typically used in medical imaging. This imaging capability has been expanded through the use of intravenously administered microbubbles as a contrast agent. These microbubbles are also used in a growing number of therapeutic applications to enhance biological effects, which include transdermal drug delivery²⁷ and transient permeabilization of the blood brain barrier²⁸.

When exposed to an ultrasound wave, gas-filled microbubbles in solution will oscillate, with the positive pressure cycle resulting in compression and the negative pressure cycle causing the bubble to expand. In an ultrasound field, microbubbles experience stable cavitation (continuous expansion and contraction) at lower pressures or inertial cavitation (violent collapse of the bubble) at higher pressures²⁹. Stable cavitation results in microstreaming; fluid movement around the bubble which induces shear stress to nearby structures (such as biofilms). At higher pressures, inertial cavitation can result in a shockwave, producing high temperatures at a small focus, and create microjets from the directional collapse of the bubble which can puncture host cells and disrupt physical barriers³⁰. Both of these pressure regimes have potential for therapeutic applications of ultrasound-mediated microbubble cavitation. Despite the potential of microbubbles to enhance drug delivery, their size (typically 1-4 micron in diameter) and short half-life once injected into solution may limit penetration and subsequent disruption of biofilms.

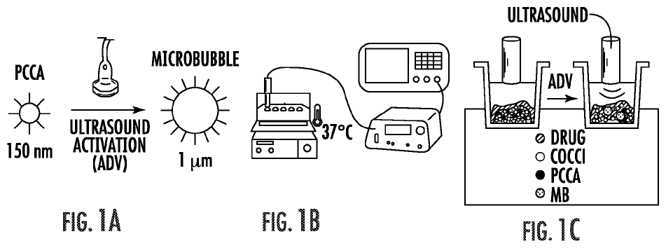

We hypothesized that phase change contrast agents (PCCA), submicron liquid particles (typically 100-400 nanometers in diameter) may be better equipped to penetrate a biofilm. Liposome encapsulated drugs (which are similar in size to PCCAs) have previously been shown to penetrate P. aeruginosa biofilms^{31,32}. In addition, unlike microbubbles, PCCA have been shown to penetrate blood clots and generate substantial internal erosion during sonothrombolysis³³. PCCAs generally consist of a liquid perfluorocarbon droplet stabilized by a phospholipid shell. With appropriate ultrasound stimulation, PCCA can convert from the liquid phase to gas, generating a microbubble in their place (FIG. 1A). This process of “acoustic droplet vaporization” (ADV) may enhance drug penetration into biofilms as microbubbles over-expand before reaching their final diameter. Prior to activation, these particles are significantly more stable in circulation than microbubbles, with pharmacokinetic half-lives on the order of 45 minutes compared to approximately 4 minutes for microbubbles^{34,35}, with the potential to diffuse into biofilms due to their small size (FIG. 1B). Additionally, with continued ultrasound application, the resulting microbubbles can generate microstreaming, shear stress and microjets as they undergo cavitation (FIGS. 1B and 1C). Typical PCCA formulations use perfluorocarbons with bulk boiling points near body temperature (e.g. dodecafluoropentane, 29° C. boiling point) and may induce undesired bioeffects as they require acoustic pressures above 3-6 MPa for ADV^{36,37}. Conversely, low boiling-point PCCA filled with octofluoropropane (−36.7° C. boiling point) can be vaporized with peak negative pressures as low as 300 kPa at 1.0 MHz frequency³⁸. These low boiling-point PCCA have been shown safe to use in vivo at moderate mechanical indices (MIs) and can be activated with clinically available hardware^{39,40}. We hypothesized that low boiling-point PCCAs, in combination with ultrasound (US-PCCA) and antibiotics that target persister cells is a novel biofilm eradication strategy.

### Results and Discussion

### Antibiotic Efficacy Against Biofilm Cells

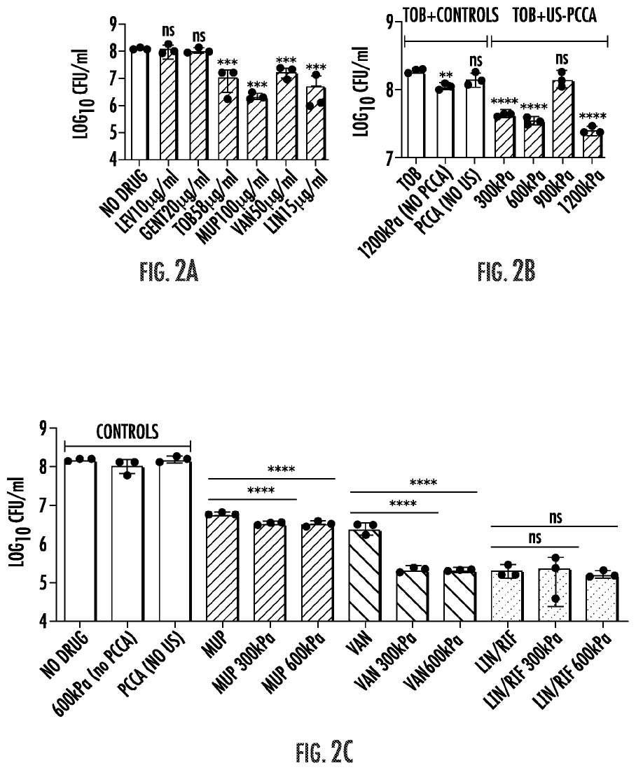

We first identified drugs with efficacy against biofilms. Antibiotics were chosen based on clinical relevance or previously reported anti-biofilm efficacy in vitro. Mature MRSA biofilms (USA300 LAC) were cultured for 24 hours in tissue culture treated plates before the addition of antibiotics. Following 24 hours of drug treatment, biofilms were washed, and survivors were enumerated by plating. Tobramycin, mupirocin, vancomycin, and linezolid all caused a significant reduction in surviving biofilm cells (FIG. 2A). In contrast, levofloxacin and gentamicin showed no efficacy against biofilms at clinically achievable concentrations found in serum (Cₘₐₓ)^{24,25} (FIG. 2B).

### Efficacy of Combined US-PCCA and Tobramycin Therapy

Next, we tested the ability of 30 second (s) US-PCCA treatment to potentiate tobramycin efficacy. Previous studies have indicated that negatively charged components of the biofilm matrix such as extracellular DNA and certain components of polysaccharides impede penetration of positively charged aminoglycosides such as tobramycin^{25,26,41}. We hypothesized that US-PCCA might improve tobramycin penetration into biofilms and increase its efficacy. Mature biofilms were washed and transferred to a custom-built temperature-controlled 37° C. water bath alignment setup (FIG. 1B). Tobramycin and PCCAs were added and ultrasound applied at a range of rarefactional pressures (300-1200 kPa). We found that tobramycin efficacy was significantly enhanced at pressures of 300, 600 and 1200 but not 900 kPa in the presence of PCCAs (FIGS. 2B and 2C). We confirmed that the addition of PCCA in the absence of ultrasound had no impact on biofilm viability. Similarly, we anticipated that ultrasound alone, in the absence of PCCA would be ineffective, however 1200 kPa did cause a small but significant reduction in surviving cells in the absence of PCCA (FIG. 2B), indicating that potentiation seen at the highest pressure (1200 kPa) may not be entirely attributable to PCCA activity, and that mechanisms other than cavitation (e.g. acoustic radiation force) may impact potentiation at this pressure. It has been previously determined that low-intensity ultrasound could potentiate gentamicin killing in P. aeruginosa biofilms without evidence of physical disruption⁴². Additionally, studies in mammalian cells show non-lethal metabolic changes and cytoskeletal rearrangement in response to low-frequency ultrasound^{43,44}. In order to investigate the potentiation effects of PCCA specifically in the regime below ultrasound-alone effects, the higher pressures (900 and 1200 kPa) were not evaluated further and the duty cycle lowered to 10% for subsequent experiments. The lower pressures, 300 and 600 kPa, in combination with PCCA were determined to be most effective at potentiating tobramycin efficacy. This is consistent with our previous findings where lower pressures (above the ADV threshold) resulted in more persistent cavitation activity during a 30 s ultrasound exposure and was consistently greatest at macromolecule drug delivery across colorectal adenocarcinoma monolayers⁴⁵.

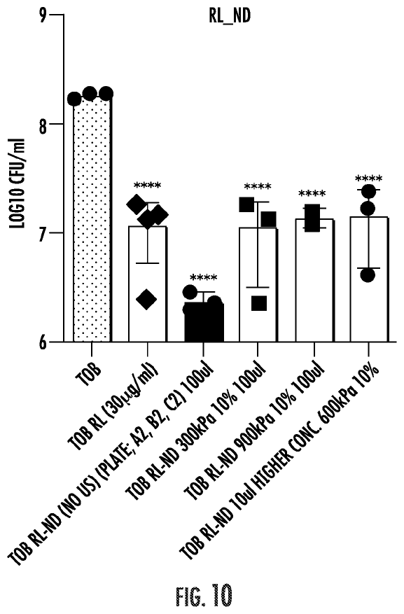

FIGS. 2A-2C illustrate that the combination of US and PCCA improves antibiotic killing of MRSA biofilms. MRSA strain LAC biofilms were cultured overnight in brain-heart infusion (BHI) media in 12-well (FIGS. 2A and 2B) or 24-well (FIG. 2C) tissue culture treated plates. Biofilms were washed and treated with antibiotics. Where indicated, plates were transferred to a custom-built temperature-controlled 37° C. water bath alignment setup. PCCA were added and 30 s ultrasound exposure was applied at indicated pressures and 20% duty cycle (FIG. 2B) or 10% duty cycle (FIG. 2C). After 24 hours, biofilms were washed, sonicated for disruption and surviving cells were enumerated by serial dilution plating. Survivors were presented as log₁₀ CFU/ml. The averages of n=3 biologically independent samples are shown. The error bars represent the standard deviation. Statistical significance was determined using a one-way analysis of variance (ANOVA) with Dunnett's (FIG. 2A) or Sidak's multiple comparison test (FIGS. 2B and 2C). **, ***, **** denotes P<0.005, P<0.0005, P<0.0001, respectively. LEV, levofloxacin; GENT, gentamicin; TOB, tobramycin; MUP, mupirocin; VAN, vancomycin; LIN, linezolid, RIF, 10 μg/ml rifampicin; ns, not significant; US-PCCA, ultrasound-stimulated phase change contrast agents.

### Efficacy of Combined US-PCCA with Clinically Relevant Antibiotic Therapy

Next, we tested the ability of US-PCCA to potentiate mupirocin, vancomycin and linezolid/rifampicin. Mupirocin is a carboxylic acid topical antibiotic commonly used to treat S. aureus infections that binds to the isoleucyl-tRNA and prevents isoleucine incorporation into proteins⁴⁶. US-PCCA caused a very slight increase in mupirocin killing (41% increase in killing) that was statistically significant but of questionable biological significance (FIG. 2C).

Vancomycin is a glycopeptide that is the frontline antibiotic to treat MRSA infections. This antibiotic acts by binding to the D-Ala-D-ala residues of the membrane bound cell wall precursor, lipid II, preventing its incorporation and stalling active peptidoglycan synthesis⁴⁷. Importantly, some studies have indicated that vancomycin penetration is impeded into biofilms²⁴. US-PCCA potentiated vancomycin killing of biofilm-associated cells by 93% (FIG. 2C), likely by improving penetration. Notably, potentiation of vancomycin was seen with the Cₘₐₓ⁴⁸ indicating that at a clinically relevant concentration, US-PCCA has the capacity to improve biofilm killing of the front-line antibiotic used to treat MRSA infections.

Linezolid is an oxazolidinone protein synthesis inhibitor that is sometimes combined with the transcriptional inhibitor, rifampicin, for the treatment of S. aureus infections^{49,50}. Linezolid/rifampicin reduced viable cells within the biofilm by almost 3-logs but was not significantly potentiated by US-PCCA (FIG. 2C). This suggests that US-PCCA has the ability to potentiate some conventional antibiotics but not others. It is possible that US-PCCA does not potentiate the killing of mupirocin and linezolid/rifampicin because the penetration of these drugs is not impeded into biofilms.

### Efficacy of Combined US-PCCA with Anti-Persister Antibiotic Therapy

Although the increased killing of biofilm-associated cells with conventional antibiotics shows promise, we hypothesized that regardless of penetration, antibiotic tolerant persister cells in the biofilm are surviving and thus impeding biofilm eradication. We predicted that utilizing US-PCCA to increase penetration of drugs active against antibiotic tolerant persister cells could further improve antibiotic therapy against biofilms.

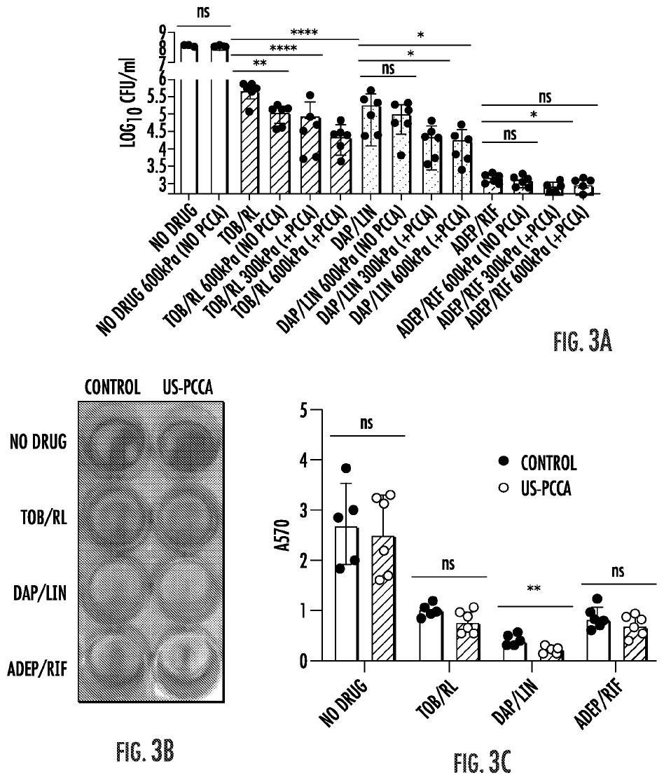

FIGS. 3A-3C illustrate that the combination of US and a PCCA improves anti-persister antibiotic therapy against MRSA biofilms. MRSA strain LAC biofilms were cultured overnight in brain-heart infusion (BHI) media in 24-well tissue culture treated plates. Biofilms were washed and treated with antibiotics and transferred to a custom-built temperature-controlled 37° C. water bath alignment setup. PCCAs were added and 30 s ultrasound exposure was applied at 300 kPa or 600 kPa (FIG. 3B and 3C) and 10% duty cycle. After 24 h, biofilms were washed, sonicated for disruption and surviving cells were enumerated by serial dilution plating (FIG. 3A) or stained with crystal violet (FIG. 3B). The averages of n=6 biologically independent samples are shown. The error bars represent the standard deviation. Statistical significance was determined using a one-way analysis of variance (ANOVA) with Dunnett's multiple comparison test (a) or multiple unpaired t-test (2-tailed) (FIG. 3C). *, **, **** denotes P<0.05, P<0.005, P<0.0001, respectively. TOB, 58 μg/ml tobramycin; RL, 30 μg/ml rhamnolipids; DAP, 100 μg/ml daptomycin; LIN, 15 μg/ml linezolid; RIF, 10 μg/ml rifampicin; ADEP, 5 μg/ml acyldepsipeptide; ns, not significant; US-PCCA, ultrasound-stimulated phase change contrast agents.

Daptomycin is a lipopeptide antibiotic which inserts into the cell membrane and disrupts fluid membrane microdomains⁵¹. Daptomycin has potent activity against recalcitrant populations of S. aureus, including biofilms^{52,53}. Daptomycin in combination with linezolid (DAP/LIN) is the treatment recommended for persistent MRSA bacteremia or vancomycin failure in the Infectious Diseases Society of America 2011 MRSA treatment guidelines⁵⁴. We found that US-PCCA increased DAP/LIN killing of MRSA biofilms by 87% and 90% at 300 kPa and 600 kPa, respectively (FIG. 3A).

Next, we wanted to investigate if US-PCCA could improve efficacy of other drugs with anti-persister activity. Acyldepsipeptides (ADEPs) are activators of the CIpP protease. We previously reported that ADEPs sterilize persisters by activating the CIpP protease and causing the cell to self-digest in an ATP-independent manner¹⁹. ADEP in combination with rifampicin reduced biofilm cells by >4-logs in 24 h. US-PCCA significantly potentiated efficacy of ADEP/RIF at 300 kPa but not 600 kPa (FIG. 3A).

Tobramycin combined with rhamnolipids (TOB/RL), has potent anti-persister activity and has eradicated several recalcitrant populations including non-respiring cells, anaerobically growing cells and small colony variants²⁰. Despite this potent anti-persister activity, TOB/RL only reduced biofilm viability by ˜3-logs²⁰. We reasoned that drug penetration might be inhibited into the biofilms and hypothesized that improving penetration could further improve efficacy against biofilms. Applying US-PCCA in combination with TOB/RL increased killing of biofilm cells by 82% and 94% at 300 kPa and 600 kPa, respectively (FIG. 3A). The reduction in viable CFUs was also associated with a decrease in biofilm biomass, as measured by crystal violet staining (FIGS. 3B and 3C).

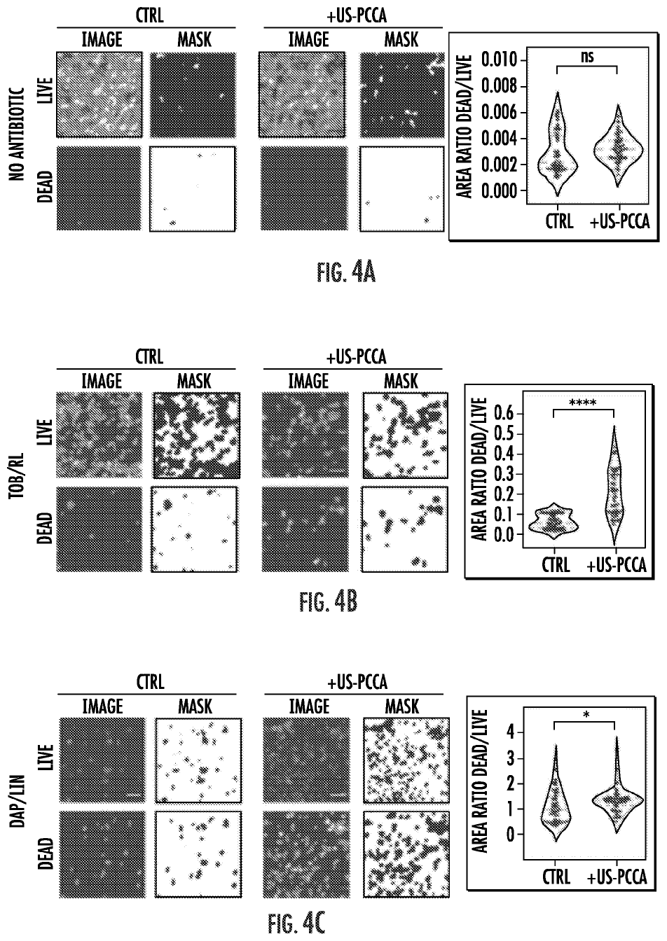

Previous studies have reported that bacteria embedded in biofilms can be coerced into a viable but non-culturable (VBNC) state in response to antibiotic pressure^{55,56}. To determine if antibiotic/ultrasound caused cell death rather than inducing a VBNC state, we examined the viability of cells within residual biofilms following antibiotic/ultrasound treatment. Biofilms were stained with LIVE/DEAD™ BacLight™ Bacterial Viability Kit and imaged with confocal laser scanning microscopy (CLSM). The viability of the biofilm was defined as a ratio between the total fluorescent signal above the threshold level covered by dead (propidium iodide positive) and total bacteria (SYTO9-positive). US-PCCA had no impact on the viability of an untreated biofilm but significantly decreased viability of the cells within biofilms treated with the anti-persister therapies tobramycin combined with rhamnolipids (TOB/RL) and daptomycin combined with linezolid (DAP/LIN) (FIGS. 4A-4C). Together this data indicates that anti-persister drugs have potent anti-biofilm activity and this can be potentiated further by improving penetration using US-PCCA.

FIGS. 4A-4C illustrate that US-PCCA in combination with anti-persister drugs reduces viability of MRSA biofilms. Biofilm viability assay in no antibiotic condition (FIG. 4A) or treated with TOB/RL (FIG. 4B) or DAP/LIN (FIG. 4C) with and without the exposure to ultrasound at 600 kPa. Upper rows show the biofilms stained with SYTO 9 representing live (total) bacteria present and their corresponding segmentation masks (black: areas covered by bacteria), while lower rows show dead bacteria within the biofilms and their segmentation masks. Scale bars indicate 5 μm. Violin and swarm plots represent the distribution of areas occupied by dead/live bacteria in independent fields of view within the biofilms (n=16 fields for each condition from 3 biological replicates each). Statistical significance of the difference between pairs was evaluated using a Student's two-sided t test. *, **** denotes P<0.05, P<0.0001, respectively. TOB, 58 μg/ml tobramycin; RL, 30 μg/ml rhamnolipids; DAP, 100 μg/ml daptomycin; LIN, 15 μg/ml linezolid; ns, not significant; US-PCCA, ultrasound-stimulated phase change contrast agents; ctrl, control. Representative images (˜4% of the area in the center) of the fields of view with values closest to the condition medians were chosen for presentation and are indicated in the swarm plots by a red point.

### Conclusions

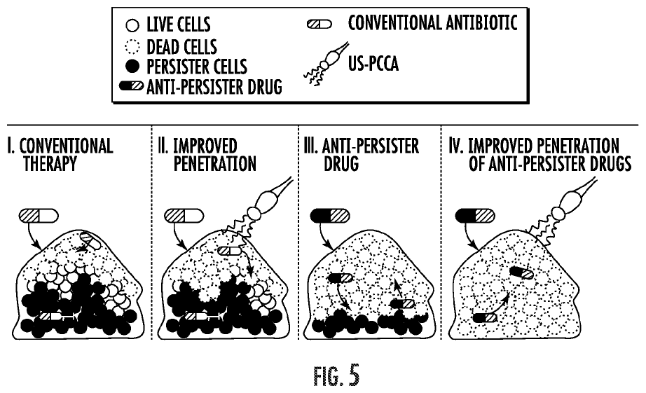

S. aureus biofilms rarely resolve with antibiotic treatment alone and usually require surgical intervention (debridement, drainage, incision)⁵⁷. Many antibiotics reduce bacterial burdens within biofilms but eradication represents an arduous challenge even in vitro^{5,15}. In this study, we combine two anti-biofilm strategies to improve therapy against biofilms (FIG. 5).

FIG. 5 is a schematic diagram representing a dual approach to improving antibiotic therapy against S. aureus biofilms. In FIG. 5, pane (I) illustrates that biofilms display remarkable tolerance to antibiotics. Susceptible cells at the biofilm periphery die (dead cells) while less metabolically active cells within the biofilm are tolerant to conventional antibiotics (persister cells). Failure to eradicate the biofilm leads to relapse in infection following removal of the antibiotic. Pane (II) of FIG. 5 illustrates that improving penetration of conventional antibiotics using US-PCCA will improve efficacy of some conventional antibiotics that do not penetrate well through the biofilm matrix. This strategy is futile as it does not improve killing of persister cells. Pane (III) of FIG. 5 illustrates that targeting biofilms with antibiotics which kill persister cells (anti-persister drug) improves efficacy but if drug penetration is impeded into the biofilm, some persister cells will remain following drug treatment and could contribute to relapsing infections. Pane (IV) of FIG. 5 illustrates that improving penetration of anti-persister drugs into the biofilm could enhance biofilm killing and reduce relapse of infection following removal of the antibiotic. The schematic diagram in FIG. 5 was created with BioRender.com.

Targeting biofilms with anti-persister drugs increases efficacy compared to conventional antibiotics (FIG. 4A). Biofilm killing by conventional antibiotics with impeded penetration is improved by US-PCCA (FIGS. 2B and 2C, FIG. 5), highlighting the therapeutic potential. US-PCCA combined with anti-persister therapies further improves biofilm killing in vitro (FIGS. 3-5). Although the clinical relevance of this strategy is not yet known, targeting two of the main drivers of biofilm antibiotic tolerance concurrently (metabolically indolent persister cells and poor drug penetration), leads to a biofilm with drastically reduced biomass and viable cells, which may facilitate subsequent immune clearance in vivo.

Antibiotic treatment failure is a complex issue that imposes a heavy burden on global public health. The last new class of antibiotics to be approved by the FDA was in 2003⁵⁸. Unlike drugs for chronic illnesses that are administered for life (e.g. heart disease, diabetes), antibiotic regimens are comparatively short, rendering the profitability of antibiotic development low⁵⁹. The void in the drug discovery pipeline makes sensitizing recalcitrant bacterial populations to already approved therapeutics a promising approach. The use of ultrasound and cavitation-enhancing agents for antibacterial applications, recently termed “sonobactericide”, was first published in 2011³⁵. While the field is still developing, a significant prospect of therapeutic ultrasound as a mechanical approach to enhance drug efficacy is its compatibility with any molecular therapeutic.

Microbubble oscillation has been shown to cause discrete morphologic changes in a P. aeruginosa biofilm⁶¹. Disruption of the physical structure of the biofilm may increase penetration depth of molecules which would otherwise be impeded. Disruption of the biofilm may have other indirect effects on drug efficacy. For example, bacterial biofilms are often hypoxic due to the diffusional distance limit of oxygen. Creating holes in the biofilm may allow oxygen penetration and stimulate the metabolic state of the residing persister cells, rendering them sensitive to antibiotics. In support of this, ultrasound in combination with microbubbles has previously been reported to alter the metabolic state of bacterial biofilms^{61,62}.

We hypothesized that PCCAs may be more efficient than microbubbles at penetrating biofilms due to their relatively small size and increased stability. PCCA have been shown to enhance cavitation erosion of blood clots for example, as they are able to penetrate and cause internal erosion in the middle of bovine clot samples from nanodroplet-mediated sonothrombolysis, whereas microbubble-mediated ultrasound generated only surface erosion³³. PCCA enhanced penetration into the biofilm matrix may therefore enhance the disruption of the biofilm matrix under ultrasound cavitation. The use of US-PCCAs has previously shown to increase vancomycin killing of MRSA biofilms⁶³. In contrast to the current study, Hu et al. used perfluoropentane as the perfluorocarbon core, which requires higher pressures than octofluoropropane to vaporize. Even in the absence of an antibiotic, US-PCCA caused a significant reduction in biofilm matrix and metabolic activity measured by three-dimensional fluorescence imaging and resazurin⁶². The difference in quantification method makes comparison with the previous study difficult (we enumerated bacterial survivors), however our results demonstrate a significant improvement in efficacy using shorter treatment times (30 s vs. 5 minutes). In addition, the low boiling point PCCAs used in the current study present the advantage that the same low-pressure ultrasound settings can be used for both ADV and subsequent microbubble cavitation. Indeed, this can be achieved with clinically available ultrasound hardware at pressures below the FDA set limits for diagnostic imaging. Additionally, PCCA formulation is a variant of FDA-approved ultrasound contrast microbubbles that have been clinically used for over 25 years in Europe, Asia and USA. This approach may improve the efficacy of existing approved drugs without the additional need for the extensive regulatory approval which accompanies a new molecule. Likewise, as it uses ultrasound parameters that are achievable with clinically available equipment, this has the potential for rapid translation to clinical practice without the need for further technological development.

The ultrasound parameters used in our study mostly varied acoustic pressure and have not yet been optimized for in vivo application. While acoustic pressure is a large contributor to PCCA activation and stimulation, other parameters of frequency, duty cycle, treatment time and PCCA concentration could be further evaluated. The selected frequency of 1 MHz is lower than the predicted resonant frequency of the resulting microbubbles. However, optimal PCCA activation parameters and optimal microbubble oscillation parameters may not be the same and will require further investigation. Ultrasound is used clinically for debridement of wounds to disperse biofilms at frequencies below 1 MHz⁶⁴. Interestingly, the lower frequency of 250 kHz was recently shown to enhance sonoporation due to large radial excursions of microbubbles well below their acoustic resonant frequency^{65,66}. Evaluation of PCCA drug potentiation using lower frequencies and higher intensities typical for this application could give further insight into clinical integration strategies. Future experiments will evaluate the potentiation of antibiotics in a S. aureus mouse skin and soft tissue infection (SSTI). For topical applications such as soft tissue infections, we believe maintaining cavitation activity for the duration of the treatment will be crucial for efficacy, as no new cavitation nuclei will be introduced as would be the case in intravenously administered PCCA (replenished by blood flow).

### METHODS

### Biofilm Assays

Biofilm assays were performed using the USA300 MRSA strain LAC. It is a highly characterized community-acquired MRSA (CA-MRSA) strain isolated in 2002 from an abscess of an inmate in Los Angeles County jail in California⁶⁷. LAC was cultured overnight (18 h) in brain heart infusion (BHI) media (Oxoid) in biological triplicates. Each culture was diluted 1:150 in fresh media and 2 or 3 ml was added to the wells of 24-well or 12-well tissue culture treated plates (Costar), respectively. Biofilms were covered with Breathe-Easier sealing strips (Sigma) and incubated at 37° C. for 24 h. Biofilms were carefully washed twice with PBS and fresh BHI media containing antibiotics was added. Biofilms were covered and incubated at 37° C. for 24 h. Biofilms were carefully washed twice with PBS before dispersal in a sonicating water bath (5 min) and vigorous pipetting. Surviving cells were enumerated by serial dilution and plating. Antibiotics were added at concentrations similar to the Cₘₐₓ in humans; 10 μg/ml levofloxacin⁶⁸ (Alfa Aesar), 20 μg/ml gentamicin⁶⁹ (Fisher BioReagents), 58 μg/ml tobramycin⁷⁰ (Sigma), 50 μg/ml vancomycin hydrochloride⁴⁸ (MP Biomedicals), 15 μg/ml linezo1id⁷¹ (Cayman Chemical), 10 μg/ml rifampicin⁷² (Fisher BioReagents), 100 μg/ml daptomycin⁷³ (Arcos Organics), with the exception of the topical antibiotic mupirocin (Sigma) (administered at 100 μg/ml) and acyldepsipeptide antibiotic (ADEP4) which was added at 10×MIC (10 μg/ml) which previously showed efficacy against S. aureus biofi1ms¹⁹. For daptomycin activity, the media was supplemented with 50 mg/L of Ca²⁺ ions. Where indicated tobramycin was supplemented with 30 μg/ml rhamnolipids²² (50/50 mix of mono- and di-rhamnolipids, Sigma). Where indicated biofilms were treated with PCCA and ultrasound. For crystal violet staining, biofilms were carefully washed twice with PBS, and dried in a 65° C. oven for 1 h. Biofilms were stained with 1 ml 0.4% crystal violet for 5 min, and washed 3× with PBS. Wells were photographed, and stain was solubilized with 2 ml 5% acetic acid and absorbance measured at 570 nm.

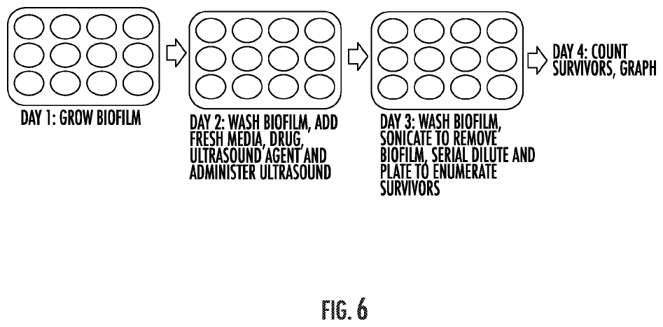

FIG. 6 illustrates an example process for growing a biofilm, treating the biofilm with a combination of a therapeutic agent, a phase change contrast agent combined with ultrasound, and determining the results. Referring to FIG. 6, in day 1, the biofilm is grown in a twelve well plate. In day 2, the biofilm is washed, fresh media is added, a therapeutic agent is added, a phase change contrast agent is added, and ultrasound is administered. In day 3, the biofilm is washed, sonicated to remove the biofilm, diluted, and plated to count survivors. In day 4, the survivors are counted, and the count is presented in graphs, such as those described above.

### PCCA Generation

Phase change contrast agents were generated as previously reported 50 [Sheeran et al.] Phase change contrast agents were generated as previously reported⁷⁴ [Sheeran et al. 2012]. Briefly, 1,2-distearoyl-sn-glycero-3-phosphocholine (DSPC) and 1,2-distearoyl-sn-glycero-3-phosphoethanolamine-N-methoxy(polyethylene-glycol)-2000 (DS PE-PEG2000) (Avanti Polar Lipids, Alabaster, Ala., USA) were dissolved in 5% glycerol, 15% propylene glycol (both from Fisher Chemical, Waltham, Mass., USA) in PBS (v/v) at a 1:9 ratio, to a total lipid concentration of 1 mg/ml. Lipid solution (1.5 ml) was dispensed into 3 ml crimp-top vials and degassed under vacuum for 30 minutes and then backfilled with octofluoropropane gas (Fluoro Med, Round Rock, Tex., USA). The vials were activated by mechanical agitation (VialMix, Bristol-Myers-Squibb, New York, N.Y., USA) to generate micron scale octofluoropropane bubbles with a lipid coat. The vials containing bubbles were cooled in an ethanol bath to −11 C. Pressurized nitrogen (45 PSI) was introduced by piercing the septa with a needle and used to condense the gaseous octofluoropropane into a liquid, creating lipid-shelled perfluorocarbon submicron droplets (PCCA). Particle size and concentration was characterized the Accusizer Nano FX (Entegris, Billerica, Mass., USA).

### Ultrasound Experiments

Ultrasound experiments were conducted in 12 or 24 well tissue culture plates using a custom fabricated water bath ultrasound alignment setup to maintain 37° C. during the experiment, similar to a design used previously with cell monolayers⁴⁵. Briefly, alignment guides were positioned above the wells to ensure reproducible transducer placement to the center of each well on top of the biofilm and 10 mm from their bottom. To limit acoustic reflections and standing waves from the bottom of the well plate, the plate was coupled to a water bath, the bottom of which was lined with acoustic absorber material. The water temperature was maintained at 37 C throughout the experiment by placing the water bath setup on a heated plate and monitored by thermocouple. A 1.0 MHz unfocused transducer (IP0102HP, Valpey Fisher Corp) was characterized via needle hydrophone and driven with an amplified 20- or 40-cycle sinusoidal signal defined on an arbitrary function generator (AFG3021C, Tektronix, Inc.; 3100LA Power Amplifier, ENI) at a pulse-repetition frequency of 5000 Hz (10% or 20% duty cycle). Peak negative pressures of 300, 600, 900 and 1200 kPa were used in the experiments. Previous experiments using octofluoropropane PCCAs at these pressures demonstrated that higher pressures significantly reduced stable and inertial cavitation persistence over a 30-s exposure whereas lower pressures sustained cavitation activity⁴⁵, indicating inertial cavitation at high pressures and a subsequent reduction of cavitation nuclei due to bubble destruction. To avoid ultrasound-alone effects on the biofilm, we focused on the lower pressures, 300 and 600 kPa, determined most effective at potentiating tobramycin efficacy with PCCA and lowered the duty cycle from 20% in FIG. 2B to 10% for subsequent figures, as this was shown to have a more modest effect in our prior work and resulted in significant drug delivery⁴⁵. Where indicated, 10 μl of PCCA was added to each well ((1.17±0.4)×10¹¹ particles/mL, 0.18 μm diameter) and mixed gently by pipetting. The transducer was positioned in the well in the media above the biofilm and ultrasound treatment was applied for 30 seconds. Following treatment, each plate was incubated at 37 C for 24 h before enumerating survivors (described in detail above).

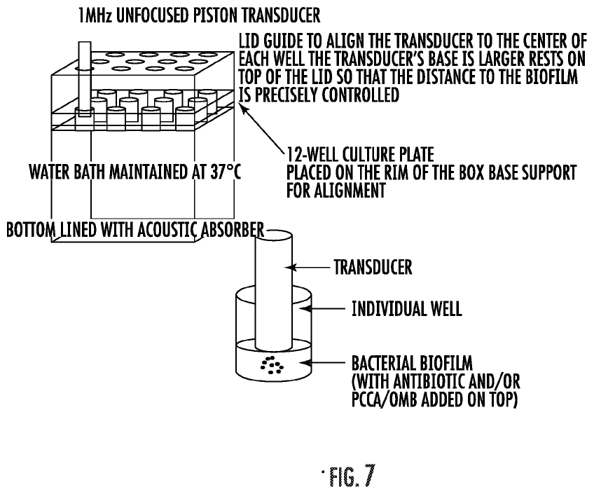

FIG. 7 is a diagram illustrating the experimental setup for sonicating the plates that include the biofilm being treated with a phase change contrast agent in combination with a therapeutic agent. In FIG. 7, a 1 MHz unfocused piston ultrasound transducer is positioned over each well. A lid is used to align the transducer with the center of each well. The transducer's base rests on top of the lid so that the distance between the transducer and the biofilm is precisely controlled. A 12 well culture plate is placed on the rim of the box lined with an acoustic absorber for alignment. FIG. 7 also illustrates the positioning of a transducer over an individual well. The transducer applies ultrasound to the biofilm located in the well from above. The biofilm includes a therapeutic agent, such as an antibiotic, with a phase change contrast agent and/or oxygen microbubbles added to the biofilm.

### Microscopy

Biofilms were cultured in 24-well plates and treated with antibiotics and US-PCCA as described above. Following 24 hours of therapy, biofilms were washed in 0.85% NaCl and stained with LIVE/DEAD™ BacLight™ Bacterial Viability Kit, for microscopy & quantitative assays (Invitrogen) for 15 min in the dark. Biofilms were washed gently in PBS and submerged in 0.5 ml PBS for imaging. Images were acquired on a Zeiss LSM 700 confocal microscope, using an LD Plan Neofluar 40×/0.6 DIC II objective, with the correction collar set to 1.0. The “live” stain was acquired with a 488 nm laser, with a 490-555 nm band pass emission filter. The “dead” stain was acquired with a 555 nm laser, with a 615 nm long pass emission filter. The multiple beam splitter position was set to 615 nm, and the microscope was operated in line-switching mode. A transmitted light image was acquired simultaneously in the 555 nm channel. For each channel, the laser power, conventional PMT master gain and digital offset were adjusted to ensure no pixels had a value of 0, and no pixels were saturated (saturation value 4095). The pinhole was set to 1 AU for the longest wavelength fluorophore (the “dead” stain), and its diameter in um was kept constant in the other channel. Images were taken with zoom set to 1.0×, 1024×1024 pixels, for a pixel size of 0.156 um. Images were averaged 4 times in line mode and unidirectional laser scanning was used. A field of 4 by 4 images was acquired centered roughly in the middle of each well, using tile scan mode without overlap. Because of imperfections in stage movement, some images overlapped slightly with their neighbors; we cropped 3.5% of each image border to avoid measuring any cells twice in our analysis. The Z plane selected for imaging was the one with the maximal number of cells, which was typically the Z plane in the sample closest to the bottom of the well. All images were acquired the same day, with the same settings. Controls with unstained samples showed that with these settings autofluorescence from bacteria or biofilms was undetectable.

### Live/Dead Quantification

Quantification of the bacteria viability from confocal images was performed using Python (3.8.3) with Numpy (1.18.5), Pandas (1.0.5), Skimage (0.16.2) and Seaborn (0.10.1) libraries. Each field within a tiled scan was considered an independent image. For each condition, three biological replicates have been imaged in sixteen fields of view (total 48 images for each condition). Images in both channels were smoothed using a gaussian filter (sigma=1) and segmented with a global threshold (100 a.u.). The viability of the biofilm was defined as a ratio between the area (or total fluorescent signal above the threshold level) covered by dead (propidium iodide positive) and live/total bacteria (SYTO9-positive). A Student's two-sided t test was performed as implemented in Python Scipy (1.5.0) stats.ttest_ind to compare control and ultrasound conditions. We observed the same results using a range of relevant threshold values as well as comparing the integrated intensity ratios above the threshold in both channels.

### Statistical Information

The averages of n=3 or n=6 biologically independent samples are shown (as indicated in the figure legends). The error bars represent the standard deviation of the mean. Statistical analysis was performed using Prism 8 (GraphPad) software. One-way ANOVA with Sidak's or Dunnett's multiple comparison test (as indicated in the figure legends). Statistical significance was defined as P<0.05.

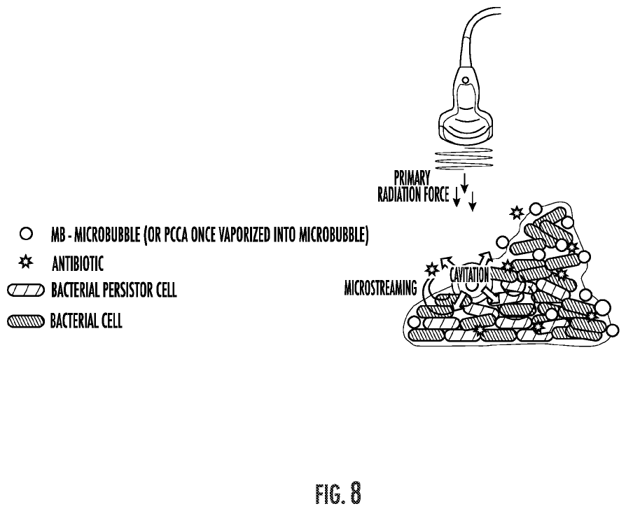

FIG. 8 is a schematic diagram illustrating the use of a cavitation enhancing agent in combination with a therapeutic agent to treat the biofilm. Referring to FIG. 8, a cavitation enhancing agent such as a phase change contrast agent, is added to biofilm. An antibiotic is also added to the biofilm. It is envisioned that in humans or animals with biofilms located in or on wounds, the antibiotic and the cavitation enhancing agent will be applied topically. It is also envisioned that the cavitation enhancing agent will be applied as phase change nanodroplets to increase penetration into the biofilm.

An ultrasound transducer applies ultrasound energy to the biofilm, which causes the cavitation enhancing agent to cavitate. If the cavitation enhancing agent is applied as phase change nanodroplets, the application of ultrasound will cause the phase of the cores of the nanodroplets to change from a liquid to a gaseous state, converting the nanodroplets into microbubbles, which oscillate in diameter. Microbubble oscillation under ultrasound (stable or inertial cavitation) helps the therapeutic agent penetrate deeper into the biofilm either directly through mechanical disruption of the biofilm matrix and also through microstreaming (driving local flow around the oscillating microbubble due to its large cyclic diameter increase and decrease period). Ultrasound also pushes in the direction of propagation, so in the case of a phase change contrast agent, due to their small nanometer scale size, will penetrate deeper into the biofilm and help drive the drug deep into the biofilm where more persister cells are located. As described above, persister cells are bacterial cells that are more tolerant to antibiotics due to their metabolically dormant state due to low oxygen and nutrients deep within the biofilm.

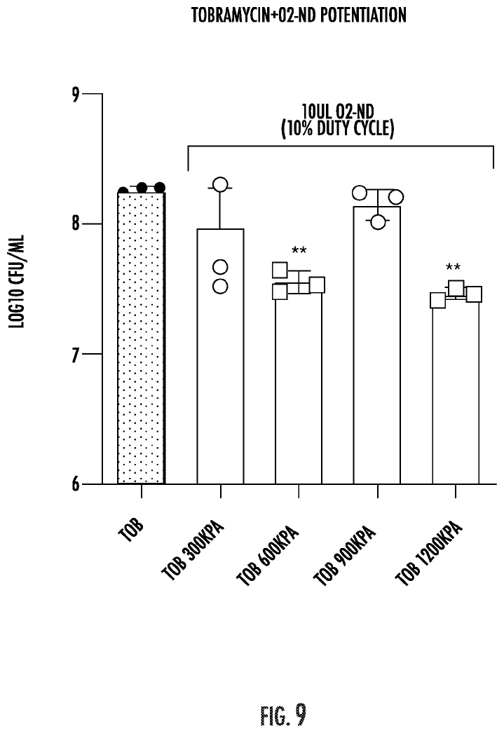

Different combinations of cavitation enhanced agents and therapeutic agents can be used to treat a biofilm. FIG. 9 illustrates the results of treating an MRSA biofilm with an oxygen nanodroplet phase change contrast agent in combination with the TOB antibiotic. The results in FIG. 9 show that the addition of the oxygen nanodroplets increase the effectiveness of the treatment over using the TOB antibiotic without the oxygen nanodroplets by reducing the number of surviving colony forming units.

FIG. 10 is a graph illustrating results of treating an MRSA biofilm with the TOB antibiotic alone and the TOB antibiotic in combination with phase change nanodroplets made from rhamnolipids. The phase change contrast agent was made using the lipid solution of rhamnolipids, gas exchange with OFP gas to form microbubbles. The microbubbles are then condensed to form nanodroplets with rhamnolipid shells and OFP liquid cores. The phase change nanodroplets were then added to the biofilm along with the TOB antibiotic. The graph in FIG. 10 shows that the administration of rhamnolipids in combination with the TOB antibiotic decreased the number of surviving CFUs per milliliter over the treatment of the biofilm with the TOB antibiotic alone.

### In Vivo Experiment and Results

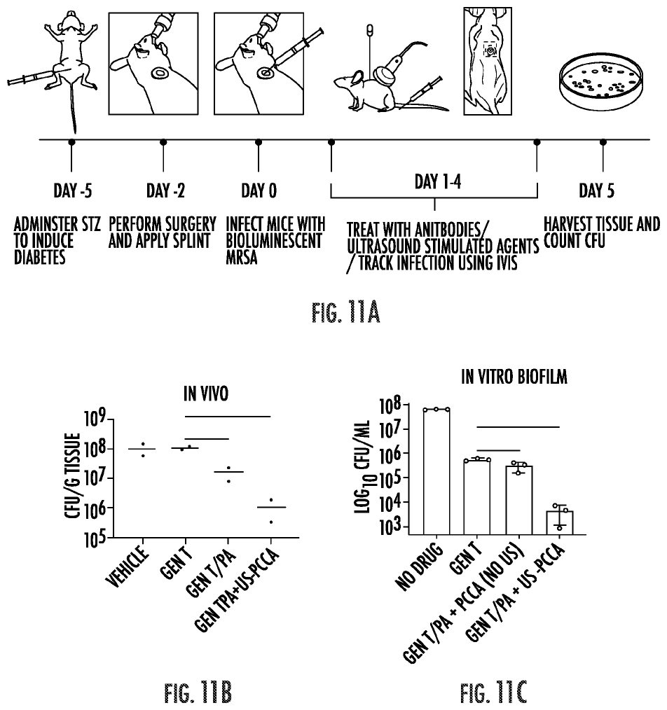

FIGS. 11A-11C illustrate that ultrasound-stimulated phase change contrast agents improve antibiotic activity against biofilms in vitro and in vivo. FIG. 11A is a schematic of our IACUC approved diabetic chronic wound model modified from Hunt et al⁷⁵. Briefly, diabetes was induced in 6-8 week old male/female SKH-1 hairless mice with a single dose of 225 mg/kg streptozocin by IP injection⁷⁶. On one side of the mouse's midline at the level of the shoulders, a 4 mm full-thickness wound was created that extends through the subcutaneous tissue including the panniculus carnosus and covered with a splint and an occlusive dressing. 2 days later, the wound was infected with ˜5e6 cfu of bioluminescent MRSA (JE2-lux)⁷⁷. Mice were treated twice daily with topical 0.1% gentamicin, 3% palmitoleic acid, US-PCCA or the vehicle for 4 days. The infection was tracked with IVIS Spectrum In Vivo Imaging System using auto settings: exposure time 5-300 s, with medium binning, 1 f/stop and open filter, and field of view C. On day 5, mice were euthanized, and the wound area was harvested, homogenized and plated to enumerate cfu. FIG. 11B illustrates results of the pilot experiment for n=2 mice/group. FIG. 11C illustrates results for MRSA biofilms were cultured overnight in brain-heart infusion (BHI) media in 24-well tissue culture treated plates. Biofilms were washed and where indicated were treated with 100 μg/ml gentamicin, 30 μg/ml palmitoleic acid, and/or ultrasound stimulated phase change contrast agents (US-PCCA) performed immediately after one of the two daily Gent/PA treatments. Each US-PCCA application consisted of 5 consecutive treatments each consisting of 50 μL PCCA solution added directly on top of the wound (topical administration) followed by 1 min of ultrasound sonication (1 MHz, 600 kPa, 10% duty cycle). After 24 hours, biofilms were washed, sonicated for disruption and surviving cells were enumerated by serial dilution plating. The averages of n=3 biologically independent samples are shown. The error bars represent the standard deviation. Statistical significance was evaluated using a One-Way Anova with Dunnett's multiple comparisons test. *, **, *** denotes P<0.05, P<0.005, and P<0.0005, respectively.

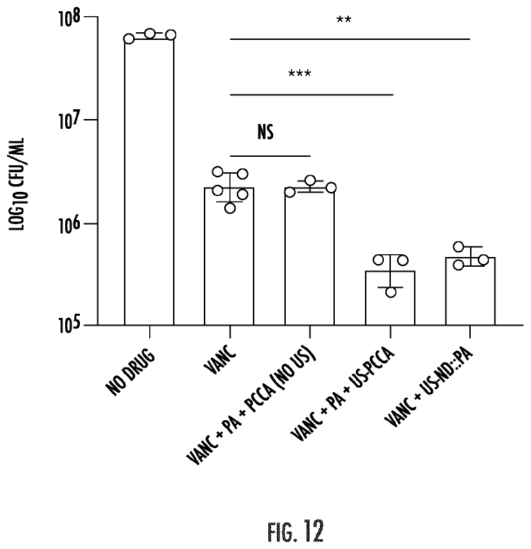

Palmitoleic acid drastically improves vancomycin killing of biofilms if penetration is increased with US-PCCA (FIG. 12). However, the utility of palmitoleic acid as an antibiotic adjuvant is limited to topical administration as it will be converted to triglycerides if used systemically⁷⁸. Formulating the nanodroplets with palmitoleic acid may expand the application of this potent antibiotic adjuvant to improve stability and penetration and allow for systemic use.

FIG. 12 illustrates that palmitoleic acid loaded nanodroplets potentiate vancomycin activity against biofilms. MRSA biofilms were cultured overnight in brain-heart infusion (BHI) media in 24-well tissue culture treated plates. Biofilms were washed and where indicated were treated with 50 μg/ml vancomycin, 30 μg/ml palmitoleic acid, or ultrasound stimulated (30 s duration, 1 MHz, 600 kPa, 10% duty cycle) phase change contrast agents (standard formulation⁷⁹) or ultrasound stimulated nanodroplets loaded with 30 μg/ml palmitoleic acid. After 24 hours, biofilms were washed, sonicated for disruption and surviving cells were enumerated by serial dilution plating. The averages of n=3 biologically independent samples are shown. The error bars represent the standard deviation. Statistical significance was evaluated using a One-Way Anova with Dunnett's multiple comparisons test. *, **, *** denotes P<0.05, P<0.005, and P<0.0005, respectively.

To manufacture the palmitoleic acid nanodroplets, a palmitoleic acid (PA) solution was prepared in propylene glycol:glycerol:PBS (15:5:80) (PGG) to a concentration of 1 mg/mL. A lipid solution was prepared in PGG by dissolving DSPE-PEG2K and DSPC (Avanti Polar Lipids, Alabaster, Ala.) at a 1:9 ratio for a total concentration of 1 mg/mL. The PA solution and lipid solution were combined at a 1:1 ratio, and 1.5 mL of the mixture was dispensed into a 3 mL glass vial with chloroform-cleaned butyl septa and sealed with a crimped cap. Vials were vacuum-degassed for 30 minutes and the headspace was filled with octafluoropropane gas (Fluoromed L. P., Round Rock, Tex.). Bubbles were generated via mechanical agitation (Vialmix, Lantheus Medical Imaging, Billerica, Mass.) for 45 seconds. PA-containing droplets were condensed by incubating in a chilled ethanol bath (−11° C.) and pressurized nitrogen gas (45 PSI) in a manner similar to Sheeran, Paul S et al. “Formulation and acoustic studies of a new phase-shift agent for diagnostic and therapeutic ultrasound.” Langmuir: the ACS journal of surfaces and colloids vol. 27,17 (2011): 10412-20. doi:10.1021/la2013705, the disclosure of which is incorporated herein by reference in its entirety.

In addition to the biofilm forming bacteria described above, the following types of bacteria may also be treated using the combinations of ultrasound, PCCAs, and therapeutic agents described herein: Staphylococcus epidermidis, Pseudomonas aeruginosa, Enterococcus faecalis, Streptococcus pyogenes, Streptococcus agalactiae, Streptococcus spp, Stenotrophomonas (Xanthomonas), and Enterobacteriaceae (Proteus mirabilis, Acinetobacter spp., Salmonella spp., Yersinia spp. E. coli, and Shigella spp.).

In addition to the biofilm infection types described above, the following biofilm infection types may also be treated using the combinations of ultrasound energy, PCCAs, and therapeutic agents described herein: complicated skin and skin structure infections (cSSSIs) including but not limited to: abscesses, burn infections, cellulitis, diabetic foot/leg ulcers, and wound infections. Other infection types that may be treated using the combinations of ultrasound, energy, PCCAs, and therapeutic agents described herein include indwelling device infections, endocarditis, osteomyelitis, lung infections, deep tissue abscesses, septic arthritis, and joint infections.

In addition to the antibiotics described above, the following can also be used in combination with ultrasound energy and PCCAs to treat infections: Amikacin, teixobactin, bacitracin, colistin, fusidic acid, and polymyxin B.

In addition to the surfactants/fatty acids described above, linoleic acid may also be used as one of the therapeutic agents.

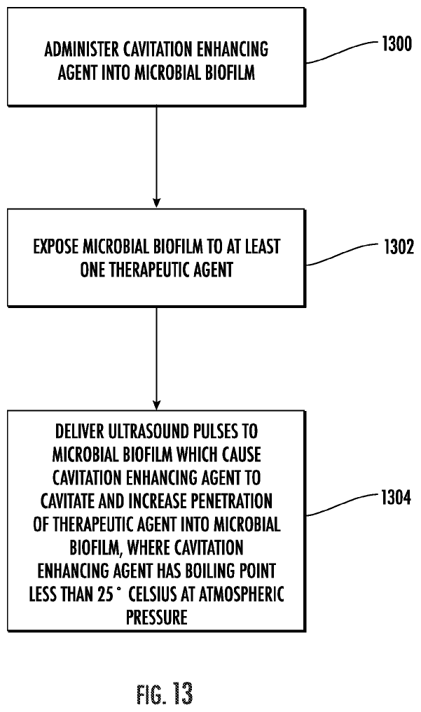

FIG. 13 is a flow chart illustrating an exemplary process for applying phase change nanodroplets to a biofilm. Referring to FIG. 13, in step 1300, the process includes administering a cavitation enhancing agent into a microbial biofilm. In one example, the cavitation enhancing agent may be phase change nanodroplets each having a core formed of a low boiling point (less than 25° C. at atmospheric pressure, where atmospheric pressure refers to pressure of one atmosphere) perfluorocarbon (or multiple of such perfluorocarbons) encapsulated in a shell. If the biofilm is located in or on a wound of a living subject, such as a mammalian subject, including, but not limited to a human or a mouse, the cavitation enhancing agent may be applied topically. If the biofilm is internal, the cavitation enhancing agent may be applied intravenously.

In step 1302, the process further includes exposing the microbial biofilm to at least one therapeutic agent. The therapeutic agent may be an antibiotic or other material used to kill microbes in the biofilm. The therapeutic agent may be applied topically, either with the cavitation enhancing agent in a separate step from the application of the cavitation enhancing agent. In one example, the cavitation enhancing agent and the therapeutic agent may be combined as a mixture and the mixture may be applied to the microbial biofilm. The therapeutic agent to which the microbial biofilm is exposed may be any of the therapeutic agents described herein. The therapeutic agent may also include and antibiotic adjuvant, such as palmitoleic acid, which enhances antibiotic activity. The therapeutic agent, including the antibiotic and the antibiotic agent may, in one example delivery mechanism, be encapsulated within individual particles of the cavitation enhancing agent.

In step 1304, the process further includes delivering ultrasound pulses to the microbial biofilm which cause the cavitation enhancing agent to cavitate; and increase penetration of the therapeutic agent into the biofilm. As indicated above, the cavitation enhancing agent may be a phase change contrast agent comprising a core including a material that has a boiling point less than 25° at atmospheric pressure. The application of ultrasound may cause the cavitation enhancing agent to form microbubbles, which oscillate, disrupt the biofilm, and increase flow of the therapeutic agent through the biofilm. Because a low boiling point material is used for the cavitation enhancing agent, the amount of ultrasound energy required to induce a phase change in the cavitation enhancing agents is reduced over that required in conventional therapies using high boiling point phase change contrast agents.

In one example of the subject matter described herein, a system for enhancing delivery of a therapeutic agent into a microbial biofilm located in or on a body of a subject is provided. The system includes an ultrasound transducer element array which delivers ultrasound energy into the microbial biofilm. The system further includes a mechanism for exposing the microbial biofilm to at least one therapeutic agent and administering a cavitation enhancing agent to the microbial biofilm located in or on the body of the subject, wherein the cavitation enhancing agent comprises a phase change contrast agent comprising a core including a material that has a boiling point less than 25° C. at atmospheric pressure. The system further includes a topical treatment device, where the ultrasound transducer element array and the mechanism for exposing and administering are components of the topical treatment device.

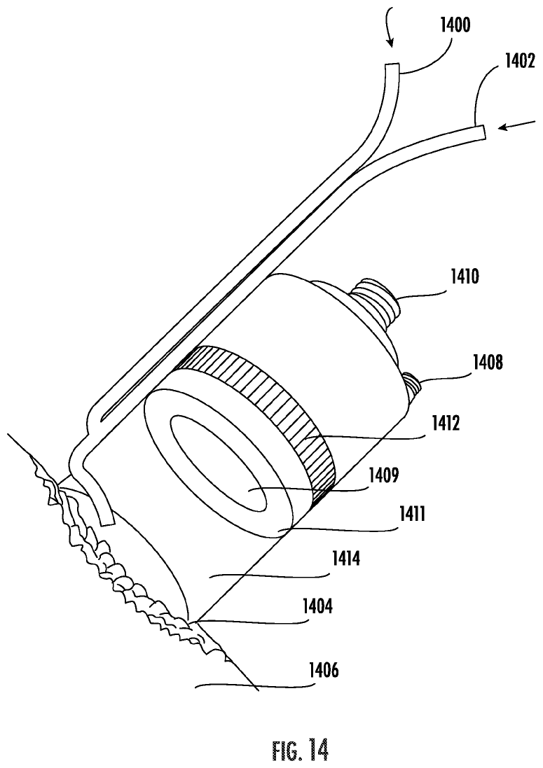

FIG. 14 is a diagram illustrating a topical treatment device for treating a microbial biofilm infection using a combination of a phase change contrast agent, a therapeutic agent, and ultrasound energy. In FIG. 14, the topical treatment device includes solution injection tubes 1400 and 1402 for injecting therapeutic agents, such as drugs, and PCCA onto or into a microbial biofilm 1404 located on an outer surface of a subject's skin 1406. Solution injection tubes 1400 and 1402 may be connected to metered injection syringe pumps (not shown in FIG. 14) to control flow rate. In addition, although FIG. 14 illustrates separate solution injection tubes 1400 and 1402 which respectively deliver therapeutic agents and PCCA to a wound area 1404 infected with a microbial biofilm, in an alternate implementation, the therapeutic agent and the PCCA can be pre-mixed and injected together into or onto wound area 1404 infected with the microbial biofilm using a single injection tube.

The topical treatment device further includes a connector 1408 for connecting to a passive cavitation detection ultrasound transducer that detects, via a passive cavitation detection ultrasound transducer element array 1409, ultrasound energy generated by vaporization and cavitation of the PCCA used to treat the wound area 1404 infected with the microbial biofilm.

The topical treatment device further includes a therapy connector 1410 for connecting a therapy ultrasound transducer array 1411 to a therapy ultrasound transducer. Therapy ultrasound transducer element array 1411 delivers the ultrasound energy to the PCCA to induce the acoustic droplet vaporization and cavitation, which disrupt the microbial biofilm. In one implementation, the therapy ultrasound transducer and the passive cavitation detection ultrasound transducer operate at different frequencies.

The topical treatment device further includes a grip holder 1412, which can be attached to a 3D motion stage for positioning and treating an entire wound area. Grip holder 1412 can also be used by a wound care specialist to manually hold the device to treat the wound area.

The topical treatment device further includes an acoustically transparent gel standoff 1414 of length the focal distance of the co-aligned transducers to couple to the wound. In the illustrated example, the topical treatment device includes a cylindrical housing, and standoff 1414 comprises a cylindrical extension from the end of the housing where ultrasound transducer arrays 1409 and 1411 are located.

In another example, the system for enhancing delivery of a therapeutic agent into a microbial biofilm located in or on a body of a subject further includes an intravascular treatment device, where the ultrasound transducer element array and the mechanism for exposing and administering are components of the intravascular treatment device.

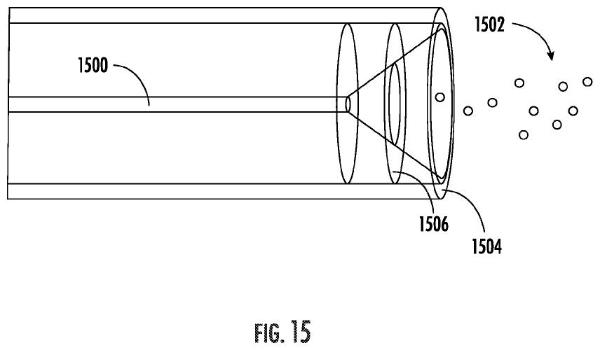

FIG. 15 is a diagram illustrating an intravascular treatment device for treating microbial biofilm infections using a combination of a phase change contrast agent, a therapeutic agent, and ultrasound energy. In FIG. 15, the treatment device comprises an intravascular catheter comprising a tube 1500 through which therapeutic agents and PCCAs 1502 are administered through or around active ultrasound elements at the end of the device that contains the active ultrasound elements. A low frequency ultrasound transducer element array 1504 provides acoustic pressure to vaporize the PCCA at the treatment target, which may be a microbial biofilm infection located within a subject's blood vessel. A co-aligned high-frequency ultrasound transducer element array 1506 is monitored for cavitation resulting from vaporization.

In another example, the system for enhancing delivery of a therapeutic agent into a microbial biofilm located in or on a body of a subject further includes an endoscopic treatment device, where the ultrasound transducer element array and the mechanism for exposing and administering are components of the endoscopic treatment device.

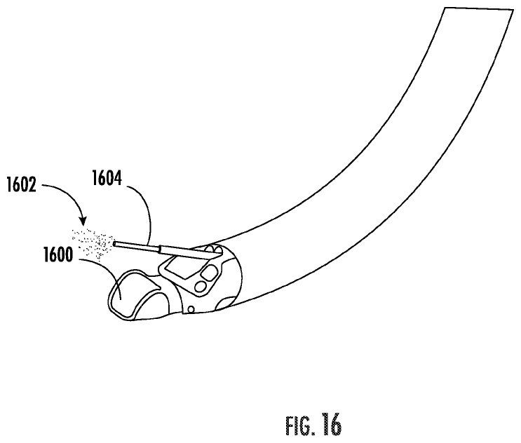

FIG. 16 is a diagram illustrating an endoscopic treatment device for treating microbial biofilm infections using a combination of a phase change contrast agent, a therapeutic agent, and ultrasound energy. In FIG. 16, the treatment device comprises an ultrasound transducer element array 1600 for delivering ultrasound energy to PCCA droplets and a therapeutic agent 1602 delivered into the body of the subject via a catheter 1604. In this example, the ultrasound transducer is capable of supplying ultrasound pressure for droplet vaporization and receiving cavitation information resulting from PA-droplet activity.

According to an aspect of the subject matter described herein, the cavitation of the cavitation enhancing agent disrupts or destroys the biofilm. For example, when ultrasound is applied to the biofilm after the cavitation enhancing agent has penetrated the biofilm, cavitating bubbles of the cavitation enhancing agent may mechanically impact the polysaccharide matrix and/or the layers of microbes in the biofilm matrix and disrupt or destroy the matrix and/or the layers of the microbes.

According to an aspect of the subject matter described herein, the biofilm comprises Pseudomonas aeruginosa (PA) or Staphylococcus aureus (SA).

According to another aspect of the subject matter described herein, the therapeutic agent comprises rhamnolipids.

According to another aspect of the subject matter described herein, the therapeutic agent comprises surfactants or fatty acids including rhamnolipids, palmitoleic acid, oleic acid, or lauric acid.

According to another aspect of the subject matter described herein, the therapeutic agent comprises oxygen gas.

According to another aspect of the subject matter described herein, the cavitation enhancing agent is a gas microbubble.

According to another aspect of the subject matter described herein, the microbubble is encapsulated within a lipid, a protein, or a surfactant.

According to another aspect of the subject matter described herein, the microbubble is encapsulated within a rhamnolipid.

According to another aspect of the subject matter described herein, the microbubble is encapsulated within a surfactant or a fatty acid, including a rhamnolipid, palm itoleic acid, oleic acid or lauric acid.

According to another aspect of the subject matter described herein, the microbubble has a core comprising a perfluorocarbon gas.

According to another aspect of the subject matter described herein, the microbubble has a core comprising oxygen gas.

According to another aspect of the subject matter described herein, the cavitation enhancing agent is a phase change contrast agent which converts from a liquid droplet to a gas microbubble when exposed to acoustic or thermal energy exceeding a threshold.

According to another aspect of the subject matter described herein, the cavitation enhancing agent comprises a core of decafluorobutane, perfluoropropane, or perfluoropentane.

According to another aspect of the subject matter described herein, the cavitation enhancing agent is a liquid core nanodroplet in a metastable state, where the perfluorocarbon core would normally be a gas in bulk state at 37° C. and atmospheric pressure.

According to another aspect of the subject matter described herein, the cavitation enhancing agent comprises oxygen in the core.

According to another aspect of the subject matter described herein, the cavitation enhancing agent comprises rhamnolipids.

According to another aspect of the subject matter described herein, the therapeutic agent comprises at least one of tobramycin, vancomycin, daptomycin, linezolid, mupirocin, levofloxacin, gentamicin, rifampicin or acyldepsipeptide antibiotic (ADEP4).

According to another aspect of the subject matter described herein, the ultrasound pulses are delivered to the phase change contrast agent in the biofilm along with the therapeutic agent within a frequency range of 20 kHz-5 MHz. In another example, the ultrasound pulses are delivered within a frequency range of 0.5-1.5 MHz.

According to another aspect of the subject matter described herein, the ultrasound pulses are transmitted to the phase change contrast agent in the biofilm along with the therapeutic agent within an acoustic pressure range of 100-2000 kPa. In another example, the ultrasound pulses are transmitted within an acoustic pressure range of 300-1200 kPa.

According to another aspect of the subject matter described herein the cavitation enhancing agent and/or the therapeutic agent are delivered superficially to a human body. In another example, the cavitation enhancing agent and the therapeutic agent may be administered internally to the human body. In another example, the cavitation enhancing agent and the therapeutic may be combined into a mixture and the mixture may be applied to a wound on the human body. In another example, the mixture may be administered intravenously into a human body or into a cavity in the human body.

The subject matter described herein also includes a system for implementing any of the methods described herein. One such system may include an ultrasound transducer which delivers ultrasound into the human body. An example of such a transducer is illustrated in FIG. 8. The system further includes a mechanism for administering at least one therapeutic agent and a cavitation enhancing agent to a microbial biofilm located in or on the human body, where the cavitation enhancing agent comprises a phase change contrast agent comprising a core including a material that has a boiling point less than 25° C. at atmospheric pressure. If the therapeutic agent and the cavitation enhancing agent are administered topically to a wound or into a body cavity of a subject, the mechanism may be a suspension, ointment, or other mixture that includes both the cavitation enhancing agent that can be applied manually by a physician. If the therapeutic agent and the cavitation enhancing agent are administered internally, the mechanism may be a syringe or a pump coupled to an intravenous port for delivering the therapeutic agent and the cavitation enhancing agent internally to a subject.

According to another aspect of the subject matter described herein, the system for administering a therapeutic agent in combination with a phase change contrast agent to a microbial biofilm may include an ultrasound coupling medium for coupling the ultrasound transducer to the human body. In one example, the ultrasound coupling medium comprises a gel. In another example, the ultrasound coupling medium comprises water.

According to another aspect of the subject matter described herein, the system for administering a therapeutic agent in combination with a phase change contrast agent may include means for mixing the therapeutic agent with the phase change contrast agent. The means for mixing may include a container or other suitable vessel for containing particles of the phase change contrast agent suspended in a liquid into which particles of the therapeutic agent can be poured. Once the two (or more) substances are combined, mixing may be effected through shaking the container, stirring the liquid, or other suitable method for making the distribution of particles of the phase change contrast agent and the therapeutic agent more uniform.

The disclosure of each of the following references is hereby incorporated herein by reference in its entirety.

### REFERENCES

1 Kourtis, A. P. et al. Vital Signs: Epidemiology and Recent Trends in Methicillin-Resistant and in Methicillin-Susceptible Staphylococcus aureus Bloodstream Infections—United States. MMWR Morb Mortal Wkly Rep 68, 214-219, doi:10.15585/mmwr.mm6809e1 (2019).
2 Boucher, H. W. & Corey, G. R. Epidemiology of methicillin-resistant Staphylococcus aureus. Clin Infect Dis 46 Suppl 5, S344-349, doi:10.1086/533590 (2008).
3 Magill, S. S. et al. Changes in Prevalence of Health Care-Associated Infections in U.S. Hospitals. N Engl J Med 379, 1732-1744, doi:10.1056/NEJMoa1801550 (2018).
4 Olaniyi, R., Pozzi, C., Grimaldi, L. & Bagnoli, F. Staphylococcus aureus-Associated Skin and Soft Tissue Infections: Anatomical Localization, Epidemiology, Therapy and Potential Prophylaxis. Curr Top Microbiol Immunol 409, 199-227, doi:10.1007/82_2016_32 (2017).
5 Bjarnsholt, T. et al. The in vivo biofilm. Trends Microbiol 21, 466-474, doi:10.1016/j.tim.2013.06.002 (2013).
6 Bjarnsholt, T. et al. Pseudomonas aeruginosa biofilms in the respiratory tract of cystic fibrosis patients. Pediatr Pulmonol 44, 547-558, doi:10.1002/ppul.21011 (2009).
7 Sonderholm, M. et al. The Consequences of Being in an Infectious Biofilm: Microenvironmental Conditions Governing Antibiotic Tolerance. Int J Mol Sci 18, doi:10.3390/ijms18122688 (2017).
8 Stewart, P. S. & Franklin, M. J. Physiological heterogeneity in biofilms. Nat Rev Microbiol 6, 199-210, doi:10.1038/nrmicro1838 (2008).
9 Stewart, P. S. & Costerton, J. W. Antibiotic resistance of bacteria in biofilms. Lancet 358, 135-138, doi:10.1016/s0140-6736(01)05321-1 (2001).
10 Lewis, K. Riddle of biofilm resistance. Antimicrob Agents Chemother 45, 999-1007, doi:10.1128/AAC.45.4.999-1007.2001 (2001).
11 Mah, T. F. & O'Toole, G. A. Mechanisms of biofilm resistance to antimicrobial agents. Trends Microbiol 9, 34-39, doi:10.1016/s0966-842x(00)01913-2 (2001).
12 Crabbe, A., Jensen, P. O., Bjarnsholt, T. & Coenye, T. Antimicrobial Tolerance and Metabolic Adaptations in Microbial Biofilms. Trends Microbiol 27, 850-863, doi:10.1016/j.tim.2019.05.003 (2019).
13 Lewis, K. Persister cells. Annu Rev Microbiol 64, 357-372, doi:10.1146/annurev.micro.112408.134306 (2010).
14 Kohanski, M. A., Dwyer, D. J. & Collins, J. J. How antibiotics kill bacteria: from targets to networks. Nat Rev Microbiol 8, 423-435, doi:10.1038/nrmicro2333 (2010).
15 Waters, E. M., Rowe, S. E., O'Gara, J. P. & Conlon, B. P. Convergence of Staphylococcus aureus Persister and Biofilm Research: Can Biofilms Be Defined as Communities of Adherent Persister Cells? PLoS Pathog 12, e1006012, doi:10.1371/journal.ppat.1006012 (2016).
16 Wood, T. K., Knabel, S. J. & Kwan, B. W. Bacterial persister cell formation and dormancy. Appl Environ Microbiol 79, 7116-7121, doi:10.1128/AEM.02636-13 (2013).
17 Lewis, K. Multidrug tolerance of biofilms and persister cells. Curr Top Microbiol Immunol 322, 107-131, doi:10.1007/978-3-540-75418-3_6 (2008).
18 Wood, T. K. Strategies for combating persister cell and biofilm infections. Microb Biotechnol 10, 1054-1056, doi:10.1111/1751-7915.12774 (2017).
19 Conlon, B. P. et al. Activated CIpP kills persisters and eradicates a chronic biofilm infection. Nature 503, 365-370, doi:10.1038/nature12790 (2013).
20 Radlinski, L. C. a. R., Sarah E. and Brzozowski, Robert and Wilkinson, Alec and Huang, Rennica and Eswara, Prahathees and Conlon, Brian P. Chemical Induction of Aminoglycoside Uptake Overcomes Antibiotic Tolerance and Resistance in Staphylococcus Aureus. CELL CHEMICAL BIOLOGY (2019).
21 Bauer, J., Siala, W., Tulkens, P. M. & Van Bambeke, F. A combined pharmacodynamic quantitative and qualitative model reveals the potent activity of daptomycin and delafloxacin against Staphylococcus aureus biofilms. Antimicrob Agents Chemother 57, 2726-2737, doi:10.1128/AAC.00181-13 (2013).
22 Radlinski, L. et al. Pseudomonas aeruginosa exoproducts determine antibiotic efficacy against Staphylococcus aureus. PLoS Biol 15, e2003981, doi:10.1371/journal.pbio.2003981 (2017).
23 Singh, R., Ray, P., Das, A. & Sharma, M. Penetration of antibiotics through Staphylococcus aureus and Staphylococcus epidermidis biofilms. J Antimicrob Chemother 65, 1955-1958, doi:10.1093/jac/dkq257 (2010).
24 Singh, R., Sahore, S., Kaur, P., Rani, A. & Ray, P. Penetration barrier contributes to bacterial biofilm-associated resistance against only select antibiotics, and exhibits genus-, strain- and antibiotic-specific differences. Pathog Dis 74, doi:10.1093/femspd/ftw056 (2016).
25 Goltermann, L. & Tolker-Nielsen, T. Importance of the Exopolysaccharide Matrix in Antimicrobial Tolerance of Pseudomonas aeruginosa Aggregates. Antimicrob Agents Chemother 61, doi:10.1128/AAC.02696-16 (2017).
26 Tseng, B. S. et al. The extracellular matrix protects Pseudomonas aeruginosa biofilms by limiting the penetration of tobramycin. Environ Microbiol 15, 2865-2878, doi:10.1111/1462-2920.12155 (2013).
27 Oberli, M. A., Schoellhammer, C. M., Langer, R. & Blankschtein, D. Ultrasound-enhanced transdermal delivery: recent advances and future challenges. Ther Deliv 5, 843-857, doi:10.4155/tde.14.32 (2014).
28 Konofagou, E. E. et al. Ultrasound-induced blood-brain barrier opening. Curr Pharm Biotechnol 13, 1332-1345, doi:10.2174/138920112800624364 (2012).
29 Church, C. C. & Carstensen, E. L. “Stable” inertial cavitation. Ultrasound Med Biol 27, 1435-1437, doi:10.1016/s0301-5629(01)00441-0 (2001).
30 Tzu-Yin, W., Wilson, K. E., Machtaler, S. & Willmann, J. K. Ultrasound and microbubble guided drug delivery: mechanistic understanding and clinical implications. Curr Pharm Biotechnol 14, 743-752, doi:10.2174/1389201014666131226114611 (2013).
31 Mugabe, C., Azghani, A. O. & Omri, A. Liposome-mediated gentamicin delivery: development and activity against resistant strains of Pseudomonas aeruginosa isolated from cystic fibrosis patients. J Antimicrob Chemother 55, 269-271, doi:10.1093/jac/dkh518 (2005).
32 Meers, P. et al. Biofilm penetration, triggered release and in vivo activity of inhaled liposomal amikacin in chronic Pseudomonas aeruginosa lung infections. J Antimicrob Chemother 61, 859-868, doi:10.1093/jac/dkn059 (2008).
33 Kim, J. et al. A Comparison of Sonothrombolysis in Aged Clots between Low-Boiling-Point Phase-Change Nanodroplets and Microbubbles of the Same Composition. Ultrasound Med Biol 46, 3059-3068, doi:10.1016/j.ultrasmedbio.2020.07.008 (2020).
34 Helfield, B. L. et al. Investigating the Accumulation of Submicron Phase-Change Droplets in Tumors. Ultrasound Med Biol 46, 2861-2870, doi:10.1016/j.ultrasmedbio.2020.06.021 (2020).
35 Fix, S. M. et al. Accelerated Clearance of Ultrasound Contrast Agents Containing Polyethylene Glycol is Associated with the Generation of Anti-Polyethylene Glycol Antibodies. Ultrasound Med Biol 44, 1266-1280, doi:10.1016/j.ultrasmedbio.2018.02.006 (2018).
36 Burgess, M. T. & Porter, T. M. Acoustic Cavitation-Mediated Delivery of Small Interfering Ribonucleic Acids with Phase-Shift Nano-Emulsions. Ultrasound Med Biol 41, 2191-2201, doi:10.1016/j.ultrasmedbio.2015.04.002 (2015).
37 Williams, R. et al. Characterization of submicron phase-change perfluorocarbon droplets for extravascular ultrasound imaging of cancer. Ultrasound Med Biol 39, 475-489, doi:10.1016/j.ultrasmedbio.2012.10.004 (2013).
38 Fix, S. M., Novell, A., Yun, Y., Dayton, P. A. & Arena, C. B. An evaluation of the sonoporation potential of low-boiling point phase-change ultrasound contrast agents in vitro. J Ther Ultrasound 5, 7, doi:10.1186/s40349-017-0085-z (2017).
39 Sheeran, P. S., Luois, S. H., Mullin, L. B., Matsunaga, T. O. & Dayton, P. A. Design of ultrasonically-activatable nanoparticles using low boiling point perfluorocarbons. Biomaterials 33, 3262-3269, doi:10.1016/j.biomaterials.2012.01.021 (2012).
40 Nyankima, A. G., Rojas, J. D., Cianciolo, R., Johnson, K. A. & Dayton, P. A. In Vivo Assessment of the Potential for Renal Bio-Effects from the Vaporization of Perfluorocarbon Phase-Change Contrast Agents. Ultrasound Med Biol 44, 368-376, doi:10.1016/j.ultrasmedbio.2017.10.016 (2018).
41 Chiang, W. C. et al. Extracellular DNA shields against aminoglycosides in Pseudomonas aeruginosa biofilms. Antimicrob Agents Chemother 57, 2352-2361, doi:10.1128/AAC.00001-13 (2013).
42 Qian, Z., Stoodley, P. & Pitt, W. G. Effect of low-intensity ultrasound upon biofilm structure from confocal scanning laser microscopy observation. Biomaterials 17, 1975-1980, doi:10.1016/0142-9612(96)00022-1 (1996).
43 Young, S. R. & Dyson, M. Macrophage responsiveness to therapeutic ultrasound. Ultrasound Med Biol 16, 809-816, doi:10.1016/0301-5629(90)90045-e (1990).
44 Zhou, S. et al. Low intensity pulsed ultrasound accelerates macrophage phagocytosis by a pathway that requires actin polymerization, Rho, and Src/MAPKs activity. Cell Signal 20, 695-704, doi:10.1016/j.cellsig.2007.12.005 (2008).
45 Fix, S. M. et al. Ultrasound-Stimulated Phase-Change Contrast Agents for Transepithelial Delivery of Macromolecules, Toward Gastrointestinal Drug Delivery. Ultrasound Med Biol 45, 1762-1776, doi:10.1016/j.ultrasmedbio.2019.02.004 (2019).
46 Hughes, J. & Mellows, G. Interaction of pseudomonic acid A with Escherichia coli B isoleucyl-tRNA synthetase. Biochem J 191, 209-219 (1980).
47 Healy, V. L., Lessard, I. A., Roper, D. I., Knox, J. R. & Walsh, C. T. Vancomycin resistance in enterococci: reprogramming of the D-ala-D-Ala ligases in bacterial peptidoglycan biosynthesis. Chem Biol 7, R109-119, doi:10.1016/s1074-5521(00)00116-2 (2000).
48 Barcia-Macay, M., Lemaire, S., Mingeot-Leclercq, M. P., Tulkens, P. M. & Van Bambeke, F. Evaluation of the extracellular and intracellular activities (human THP-1 macrophages) of telavancin versus vancomycin against methicillin-susceptible, methicillin-resistant, vancomycin-intermediate and vancomycin-resistant Staphylococcus aureus. J Antimicrob Chemother 58, 1177-1184, doi:10.1093/jac/dk1424 (2006).
49 Schwalm, J. D., El-Helou, P. & Lee, C. H. Clinical outcome with oral linezolid and rifampin following recurrent methicillin-resistant Staphylococcus aureus bacteremia despite prolonged vancomycin treatment. Can J Infect Dis 15, 97-100, doi:10.1155/2004/768765 (2004).
50 Hashimoto, S. et al. Effect of coadministration of rifampicin on the pharmacokinetics of linezolid: clinical and animal studies. J Pharm Health Care Sci 4, 27, doi:10.1186/s40780-018-0123-1 (2018).
51 Grein, F. et al. Ca(2+)-Daptomycin targets cell wall biosynthesis by forming a tripartite complex with undecaprenyl-coupled intermediates and membrane lipids. Nat Commun 11, 1455, doi:10.1038/s41467-020-15257-1 (2020).
52 Lechner, S., Lewis, K. & Bertram, R. Staphylococcus aureus persisters tolerant to bactericidal antibiotics. J Mol Microbiol Biotechnol 22, 235-244, doi:10.1159/000342449 (2012).
53 Raad, I. et al. Comparative activities of daptomycin, linezolid, and tigecycline against catheter-related methicillin-resistant Staphylococcus bacteremic isolates embedded in biofilm. Antimicrob Agents Chemother 51, 1656-1660, doi:10.1128/AAC.00350-06 (2007).
54 Liu, C. et al. Clinical practice guidelines by the infectious diseases society of america for the treatment of methicillin-resistant Staphylococcus aureus infections in adults and children: executive summary. Clin Infect Dis 52, 285-292, doi:10.1093/cid/cir034 (2011).
55 Pasquaroli, S. et al. Role of daptomycin in the induction and persistence of the viable but non-culturable state of Staphylococcus aureus biofilms. Pathogens 3, 759-768, doi:10.3390/pathogens3030759 (2014).
56 Pasquaroli, S. et al. Antibiotic pressure can induce the viable but non-culturable state in Staphylococcus aureus growing in biofilms. J Antimicrob Chemother 68, 1812-1817, doi:10.1093/jac/dkt086 (2013).
57 Negut, I., Grumezescu, V. & Grumezescu, A. M. Treatment Strategies for Infected Wounds. Molecules 23, doi:10.3390/molecules23092392 (2018).
58 Fair, R. J. & Tor, Y. Antibiotics and bacterial resistance in the 21st century. Perspect Medicin Chem 6, 25-64, doi:10.4137/PMC.S14459 (2014).
59 Bartlett, J. G., Gilbert, D. N. & Spellberg, B. Seven ways to preserve the miracle of antibiotics. Clin Infect Dis 56, 1445-1450, doi:10.1093/cid/cit070 (2013).
60 Lattwein, K. R. et al. Sonobactericide: An Emerging Treatment Strategy for Bacterial Infections. Ultrasound Med Biol 46, 193-215, doi:10.1016/j.ultrasmedbio.2019.09.011 (2020).
61 Ronan, E., Edjiu, N., Kroukamp, O., Wolfaardt, G. & Karshafian, R. USMB-induced synergistic enhancement of aminoglycoside antibiotics in biofilms. Ultrasonics 69, 182-190, doi:10.1016/j.ultras.2016.03.017 (2016).
62 Hu, J. et al. The synergistic bactericidal effect of vancomycin on UTMD treated biofilm involves damage to bacterial cells and enhancement of metabolic activities. Sci Rep 8, 192, doi:10.1038/s41598-017-18496-3 (2018).
63 Guo, H. et al. Stimulated phase-shift acoustic nanodroplets enhance vancomycin efficacy against methicillin-resistant Staphylococcus aureus biofilms. Int J Nanomedicine 12, 4679-4690, doi:10.2147/IJN.S134525 (2017).
64 Chang, Y. R., Perry, J. & Cross, K. Low-Frequency Ultrasound Debridement in Chronic Wound Healing: A Systematic Review of Current Evidence. Plast Surg (Oakv) 25, 21-26, doi:10.1177/2292550317693813 (2017).
65 Ilovitsh, T. et al. Low-frequency ultrasound-mediated cytokine transfection enhances T cell recruitment at local and distant tumor sites. Proc Natl Acad Sci USA 117, 12674-12685, doi:10.1073/pnas.1914906117 (2020).
66 Ilovitsh, T. et al. Enhanced microbubble contrast agent oscillation following 250 kHz insonation. Sci Rep 8, 16347, doi:10.1038/s41598-018-34494-5 (2018).
67 Kennedy, A. D. et al. Epidemic community-associated methicillin-resistant Staphylococcus aureus: recent clonal expansion and diversification. Proc Natl Acad Sci USA 105, 1327-1332, doi:10.1073/pnas.0710217105 (2008).
68 Cao, G. et al. Pharmacokinetics and pharmacodynamics of levofloxacin injection in healthy Chinese volunteers and dosing regimen optimization. J Clin Pharm Ther 38, 394-400, doi:10.1111/jcpt.12074 (2013).
69 Lanao, J. M. et al. Pharmacokinetic basis for the use of extended interval dosage regimens of gentamicin in neonates. J Antimicrob Chemother 54, 193-198, doi:10.1093/jac/dkh261 (2004).
70 Burkhardt, O. et al. Once-daily tobramycin in cystic fibrosis: better for clinical outcome than thrice-daily tobramycin but more resistance development? J Antimicrob Chemother 58, 822-829, doi:10.1093/jac/dk1328 (2006).
71 MacGowan, A. P. Pharmacokinetic and pharmacodynamic profile of linezolid in healthy volunteers and patients with Gram-positive infections. J Antimicrob Chemother 51 Suppl 2, ii17-25, doi:10.1093/jac/dkg248 (2003).
72 Peloquin, C. A. et al. Population pharmacokinetic modeling of isoniazid, rifam pin, and pyrazinamide. Antimicrob Agents Chemother 41, 2670-2679 (1997).
73 Benvenuto, M., Benziger, D. P., Yankelev, S. & Vigliani, G. Pharmacokinetics and tolerability of daptomycin at doses up to 12 milligrams per kilogram of body weight once daily in healthy volunteers. Antimicrob Agents Chemother 50, 3245-3249, doi:10.1128/AAC.00247-06 (2006).
74 Sheeran, P. S., Luois, S., Dayton, P. A. & Matsunaga, T. O. Formulation and acoustic studies of a new phase-shift agent for diagnostic and therapeutic ultrasound. Langmuir 27, 10412-10420, doi:10.1021/la2013705 (2011).
75 Hunt, A. M. A. et al. Come to the Light Side: In Vivo Monitoring of Pseudomonas aeruginosa Biofilm Infections in Chronic Wounds in a Diabetic Hairless Murine Model. J Vis Exp, doi:10.3791/55991 (2017).
76 Thurlow, L. R., Stephens, A. C., Hurley, K. E. & Richardson, A. R. Lack of nutritional immunity in diabetic skin infections promotes Staphylococcus aureus virulence. Sci Adv 6, doi:10.1126/sciadv.abc5569 (2020).
77 Liu, H. et al. Staphylococcus aureus Epicutaneous Exposure Drives Skin Inflammation via IL-36-Mediated T Cell Responses. Cell Host Microbe 22, 653-666 e655, doi:10.1016/j.chom.2017.10.006 (2017).
78 Santos, M., Friedberg, S. J., Kudzma, D. J. & Tjoa, L. T. Conversion of free fatty acids to triglycerides. Determination in obstructive vs hepatocellular jaundice, and cirrhosis. Arch Intern Med 134, 457-460 (1974).
79 Durham, P. G. S., A. E.; Beam, J. E.; Kedziora, K. M.; Dayton, P. A.; Conlon, B. P. Papadopoulou, V.; Rowe, S. E. Harnessing Ultrasound-Stimulated Phase Change Contrast Agents to Improve Antibiotic Efficacy Against Methicillin-Resistant Staphylococcus aureus Biofilms. Biofilm, doi:org/10.1016/j.bioflm.2021.100049 (2021).

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A method of enhancing delivery of a therapeutic agent into a microbial biofilm, the method comprising:
administering a cavitation enhancing agent into the microbial biofilm;
exposing the microbial biofilm to at least one therapeutic agent; and
delivering ultrasound pulses to the microbial biofilm which cause the cavitation enhancing agent to cavitate and increase penetration of the at least one therapeutic agent into the biofilm, wherein the cavitation enhancing agent comprises a phase change contrast agent comprising a core including a material that has a boiling point less than 25° C. at atmospheric pressure.

2. The method of claim 1, wherein the cavitation disrupts or destroys the biofilm.

3. The method of claim 1, wherein the at least one therapeutic agent comprises an antibiotic.

4. The method of claim 1, wherein the biofilm comprises Pseudomonas aeruginosa (PA) or Staphylococcus aureus (SA).

5. The method of claim 1, wherein the at least one therapeutic agent comprises rhamnolipids.

6. The method of claim 1, wherein the at least one therapeutic agent comprises surfactants or fatty acids including rhamnolipids, palmitoleic acid, oleic acid, or lauric acid.

7. The method of claim 1, wherein the at least one therapeutic agent comprises oxygen gas.

8. The method of claim 1, wherein the cavitation enhancing agent is a gas microbubble.

9. The method of claim 8, wherein the microbubble is encapsulated within a lipid, a protein, or a surfactant.

10. The method of claim 8, wherein the microbubble is encapsulated within a rhamnolipid.

11. The method of claim 8, wherein the microbubble is encapsulated within a surfactant or a fatty acid, including a rhamnolipid, palmitoleic acid, oleic acid or lauric acid.

12. The method of claim 8, wherein the microbubble has a core comprising a perfluorocarbon gas.

13. The method of claim 8, wherein the microbubble has a core comprising oxygen gas.

14. The method of claim 1, wherein the cavitation enhancing agent is a phase change contrast agent which converts from a liquid droplet to a gas microbubble when exposed to acoustic or thermal energy exceeding a threshold.

15. The method of claim 14, wherein the cavitation enhancing agent comprises a core of a perfluorocarbon including one or more of decafluorobutane, perfluoropropane, and perfluoropentane.

16. The method of claim 15, wherein the cavitation enhancing agent is a nanodroplet and the core comprises a liquid in a metastable state, and the core comprises a material that would normally be a gas in bulk state at 37° C. and standard atmospheric pressure.

17. The method of claim 14, wherein the cavitation enhancing agent comprises oxygen in a core.

18. The method of claim 14, wherein the cavitation enhancing agent comprises rhamnolipids.

19. The method of claim 1, wherein the at least one therapeutic agent comprises at least one of tobramycin, vancomycin, daptomycin, linezolid, mupirocin, levofloxacin, gentamicin, rifampicin or acyldepsipeptide antibiotic (ADEP4).

20. The method of claim 1, wherein the ultrasound pulses are delivered within a frequency range of 20 kHz-5 MHz.

21. The method of claim 1, wherein the ultrasound pulses are delivered within a frequency range of 0.5-1.5 MHz.

22. The method of claim 1, wherein the ultrasound pulses are transmitted within an acoustic pressure range of 100-2000 kPa.

23. The method of claim 1, wherein the ultrasound pulses are transmitted within an acoustic pressure range of 300-1200 kPa.

24. The method of claim 1, wherein the cavitation enhancing agent and/or the at least one therapeutic agent are delivered superficially to a human body.

25. The method of claim 1, wherein administering the cavitation enhancing agent and exposing the biofilm to the at least one therapeutic agent includes administering the cavitation enhancing agent or the at least one therapeutic agent internally to a human body.

26. The method of claim 1, wherein administering the cavitation enhancing agent and exposing the biofilm to the at least one therapeutic agent includes combining the cavitation enhancing agent and the therapeutic agent into a mixture and applying the mixture to a wound on a human body.

27. The method of claim 1, wherein administering the cavitation enhancing agent and exposing the biofilm to the at least one therapeutic agent includes combining the cavitation enhancing agent and the at least one therapeutic agent into a mixture and administering the mixture intravenously into a human body.

28. The method of claim 1, wherein administering the cavitation enhancing agent and exposing the biofilm to the at least one therapeutic agent includes combining the cavitation enhancing agent and the at least one therapeutic agent into a mixture and administering the mixture into a cavity in a human body.

29. The method of claim 1, wherein the microbial biofilm is located in or on a body of a living subject.

30. The method of claim 29, wherein the at least one therapeutic agent comprises an antibiotic and an antibiotic adjuvant.

31. The method of claim 30, wherein the antibiotic comprises gentamicin and the antibiotic adjuvant comprises palmitoleic acid.

32. A system for implementing the method of any one of claims 1-31.

33. A system for enhancing delivery of a therapeutic agent into a microbial biofilm located in or on a body of a subject, the system comprising:
an ultrasound transducer element array which delivers ultrasound energy into the microbial biofilm; and
a mechanism for exposing the microbial biofilm to at least one therapeutic agent and administering a cavitation enhancing agent to the microbial biofilm located in or on the body of the subject, wherein the cavitation enhancing agent comprises a phase change contrast agent comprising a core including a material that has a boiling point less than 25° C. at atmospheric pressure.

34. The system of claim 33, comprising an ultrasound coupling medium for coupling the ultrasound transducer to the body of the subject.

35. The system of claim 34, wherein the ultrasound coupling medium comprises a gel.

36. The system of claim 34, wherein the ultrasound coupling medium comprises water.

37. The system of claim 33, wherein the mechanism for exposing and administering includes means for applying the cavitation enhancing agent and the at least one therapeutic agent to a wound on the body of the subject.

38. The system of claim 33, wherein the mechanism for exposing and administering includes means for mixing the cavitation enhancing agent and at least one therapeutic agent into a mixture and for administering the mixture topically to a wound on the body of the subject.

39. The system of claim 33, wherein the mechanism for exposing and administering includes means for mixing the cavitation enhancing agent and at least one therapeutic agent into a mixture and for administering the mixture intravenously into the body of the subject.

40. The system of claim 33, wherein the mechanism for exposing and administering includes means for combining the cavitation enhancing agent and the at least one therapeutic agent into a mixture and for administering the mixture into a cavity in the body of the subject.

41. The system of claim 33, wherein the subject comprises a living subject.

42. The system of claim 41, wherein the at least one therapeutic agent comprises an antibiotic and an antibiotic adjuvant.

43. The system of claim 42, wherein the antibiotic comprises gentamicin and the antibiotic adjuvant comprises palm itoleic acid.

44. The system of claim 41, wherein the subject comprises a human subject.

45. The system of claim 33 comprising a topical treatment device, wherein the ultrasound transducer element array and the mechanism for administering and exposing are components of the topical treatment device, which delivers the at least one therapeutic agent and administers the cavitation enhancing agent to the microbial biofilm, which is located on the skin of the subject, and the ultrasound transducer element array delivers the ultrasound energy to the cavitation enhancing agent when the cavitation enhancing agent is located in or on the microbial biofilm.

46. The system of claim 33 comprising an intravascular treatment device, wherein the ultrasound transducer element array and the mechanism for administering and exposing are components of the intravascular treatment device, which delivers the at least one therapeutic agent and administers the cavitation enhancing agent to the microbial biofilm, which is located within a blood vessel the subject, and the ultrasound transducer element array delivers the ultrasound energy to the cavitation enhancing agent when the cavitation enhancing agent is located in or on the microbial biofilm.

47. The system of claim 33 comprising an endoscopic treatment device, wherein the ultrasound transducer element array and the mechanism for administering and exposing are components of the endoscopic treatment device, which delivers the at least one therapeutic agent and administers the cavitation enhancing agent to the microbial biofilm, which is located within the body of the subject, and the ultrasound transducer element array delivers the ultrasound energy to the cavitation enhancing agent when the cavitation enhancing agent is located in or on the microbial biofilm.
